(19) United States
(12) Patent Application Publication
(10) Pub. No.: US 2005/0191743 A1
(43) Pub. Date: Sep. 1, 2005

(54) Three-dimensional peripheral lymphoid organ cell cultures

(76) Inventors: J.H. David Wu, Pittsford, NY (US); Andrea Bottaro, Rochester, NY (US)

Correspondence Address: Nixon Peabody LLP, Clinton Square, P.O. Box 31051, Rochester, NY, 14603-1051, US

(21) Appl. No.: 10/679,184

(22) Filed: Oct. 3, 2003

Related U.S. Application Data
(60) Provisional application No. 60/415,944, filed on Oct. 3, 2002.

(51) Int. Cl. A61K 39/395; C12N 5/08

(52) U.S. Cl. 435/366

(57) ABSTRACT

The present invention relates to a method of culturing peripheral lymphoid organ cells on a three-dimensional scaffolding which is covered or surrounded with culture medium, under conditions effective to generate and maintain mature and functional lymphoid cells, where the three-dimensional scaffolding allows cells in the culture medium to have cell to cell contact in three dimensions. The present invention also provides methods of screening for vaccine candidates for efficacy in eliciting an immune response, identifying genes or proteins which are related to peripheral lymphoid organ cell formation or function, screening for drugs effecting peripheral lymphoid organ cell maturation, treating a patient for a disease condition using antigen-specific lymphoid cells, and effecting gene expression of peripheral lymphoid organ cells.

This application claims the benefit of U.S. Provisional Patent Application Ser. No. 60/415,944, filed Oct. 3, 2002.

The subject matter of this application was made with support from the National Aeronautics and Space Administration, NASA Grant No. NAG 9-1360 and NIH-NIAID Grant No. 1 R21 AI47988-01A1. The U.S. Government may have certain rights.

### FIELD OF THE INVENTION

The present invention relates to the field of cell culture, and, in particular, to methods and compositions related to cultured immune system cells derived from peripheral lymphoid organ cells.

### BACKGROUND OF THE INVENTION

Peripheral lymphoid organs represent the principal sites of adaptive immune responses and their complex anatomy reflects the presence of well-defined subregions enriched for specific immune system cell subsets. As a model peripheral lymphoid organ, the ultrastructure of the spleen is reviewed here (see also review in Tarlinton D., “Germinal Centers: Form and Function,” Curr. Opin. Immunol. 10:245-251 (1998)).

In the mammalian spleen, leukocytes localize principally within the so-called white pulp, while the red pulp is predominantly involved in the trafficking and processing of erythrocytes. The white pulp is organized around the afferent vases from which leukocytes enter the spleen, and can be subdivided into 3 main regions, as shown in FIG. 1. The first area is formed by the follicles, which are highly enriched for mature B cells expressing high surface IgD levels and low IgM (follicular B, FB, or B2 cells), as well as follicular dendritic cells (FDC), also involved in antigen capture and presentation. The marginal zone (MZ) surrounding the follicle is enriched in a population of B lymphocytes characterized by high levels of expression of IgM and of the CD21 marker; MZ B cells are thought to be involved in primary responses and particularly in responses to T cell helper-independent antigens. Adjacent to the B cell follicle is a T cell-rich zone, the periarterioral lymphoid sheets (PALS), which also contains a population of specialized antigen-presenting cells, the dendritic cells (DC). During a humoral immune response, specialized structures called germinal centers (GC) appear in the follicular area: they are the principal sites for several crucial activities of activated B cells: clonal expansion, affinity maturation (the increase in antibody affinity to antigen following hypermutation of antibody genes), generation of secondary Ig isotypes (class switching) (Tarlinton D., “Germinal Centers: Form and Function,” Curr. Opin. Immunol. 10:245-251 (1998); and MacLennan I. C., “Germinal Centers,” Annu. Rev. Immunol. 12:117-139 (1994)). Also, probably in the GC, the crucial terminal B cell differentiation decision is made between the path to long-lived memory B cells and that to antibody-secreting plasma cells (Tarlinton D., “Germinal Centers: Form and Function,” Curr. Opin. Immunol. 10:245-251 (1998); MacLennan I. C., “Germinal Centers,” Annu. Rev. Immunol. 12:117-139 (1994)). The basic organization of the spleen in segregated B and T cell areas is replicated in lymph nodes, with the exception of the absence of marginal zones.

The highly structured anatomy of peripheral lymphoid organs reflects complex homing, cell-cell interaction and adhesion processes that are essential for immune system function (Cyster et al., “Follicular Stromal Cells and Lymphocyte Homing to Follicles,” Immunol. Rev. 176:181-193 (2000); Matsumoto, “Role of TNF Ligand and Receptor Family in the Lymphoid Organogenesis Defined by Gene Targeting,” J. Med. Invest. 46:141-150 (1999)). Structural disruption of peripheral lymphoid organs, as in the case of mutants for certain TNF-family factors and their receptors, results in an inability to mount fully functional immune responses (Le Hir et al., “Differentiation of Follicular Dendritic Cells and Full Antibody Responses Require Tumor Necrosis Factor Receptor-1 Signaling,” J. Exp. Med. 183:2367-2372 (1996); Tkachuk et al., “Crucial Role of Tumor Necrosis Factor Receptor 1 Expression on Nonhematopoietic Cells for B Cell Localization Within the Splenic White Pulp,” J. Exp. Med. 187:469-477 (1998); Matsumoto et al., “Role of Lymphotoxin and the Type I TNF Receptor in the Formation of Germinal Centers,” Science 271:1289-1291 (1996), Cyster et al., “Follicular Stromal Cells and Lymphocyte Homing to Follicles,” Immunol. Rev. 176:181-193 (2000); Matsumoto, “Role of TNF Ligand and Receptor Family in the Lymphoid Organogenesis Defined by Gene Targeting,” J. Med. Invest. 46:141-150 (1999)).

During de novo lymphoid organogenesis, hematopoietic fetal liver-derived non-T/non-B precursors with CD45+, CD4+, CD3− phenotype represent one of the earliest colonizing cell of lymph nodes (Mebius et al., “Developing Lymph Nodes Collect CD4+ CD3− LTβ+Cells That Can Differentiate to APC, NK Cells and Follicular Cells but not T or B Cells,” Immunity 7:493-504 (1997); Honda et al., “Molecular Basis for Hematopoietic/Mesenchymal Interaction During Initiation of Peyer's Patch Organogenesis,” J. Exp. Med. 193:621-630 (2001); Cupedo et al., “The Role of CD45+CD4+ CD3—Cells in Lymphoid Organ Development,” Immunological Review 189:41-50 (2002)). These cells are believed to be the main cellular source of lymphotoxin (LT) β1β2, a cytokine critical for the formation of organized peripheral lymphoid tissues (review in Matsumoto, “Role of TNF Ligand and Receptor Family in the Lymphoid Organogenesis Defined by Gene Targeting,” J. Med. Invest. 46:141-150 (1999); Mebius, “Organogenesis of Lymphoid Tissues,” Nat. Rev. Immunol. 3:292-303 (2003)). Interestingly, similar CD4+, CD3− cells also play an important role in T-B cell cooperation during primary and memory phases of the immune response in the adult organism (Kim et al., “CD4+ CD3− Accessory Cells Costimulate Primed CD4 T Cells Through OX40 and CD30 at Sites Where T Cells Collaborate with B Cells,” Immunity 18:643-654 (2003)). Signaling by LTα1β2 through the LTβ receptor (LTβR) expressed by stromal cells leads to up-regulation of adhesion molecules and production of chemokines, chiefly CXCL13. These, in turn, induce clustering of stromal and hematopoietic cells, initiating the organogenesis process (review in Mebius, “Organogenesis of Lymphoid Tissues,” Nat. Rev. Immunol. 3:292-303 (2003)). CXCL13 plays an essential role in these events, as no follicles are formed in lymphoid organs of CXCL13 mutant mice (Ansel et al., “A Chemokine-Driven Positive Feedback Loop Organizes Lymphoid Follicles,” Nature 406:309-314 (2000)). These early events are followed by further colonization of lymph node structures with diverse hematopoietic subsets (B and T cells, dendritic cells, and macrophages), which also get intimately involved in the organogenesis process (Forster et al., “A Putative Chemokine Receptor, BLR1, Directs B Cell Migration to Defined Lymphoid Organs and Specific Anatomic Compartments of the Spleen,” Cell 87:1037-1047 (1996); Lorenz et al., “Isolated Lymphoid Follicle Formation is Inducible and Dependent Upon Lymphotoxin-Sufficient B Lymphocytes, Lymphotoxin β Receptor, and TNF Receptor I Function,” J. Immunol. 170:5475-5482 (2003)). LTα1β2 exerts further control on the organization and maintenance of the lymphoid organ structure (review in Matsumoto, “Role of TNF Ligand and Receptor Family in the Lymphoid Organogenesis Defined by Gene Targeting,” J. Med. Invest. 46:141-150 (1999); and Mebius, “Organogenesis of Lymphoid Tissues,” Nat. Rev. Immunol. 3:292-303 (2003)).

Importantly, many of the microenvironmental signals required for lymphoid organogenesis also appear to be necessary and sufficient during de novo formation of tertiary lymphoid structures (lymphoid neogenesis), which occurs in some inflammatory processes of infectious or autoimmune origin (Hjelmstrom, “Lymphoid Neogenesis: De Novo Formation of Lymphoid Tissue in Chronic Inflammation Through Expression of Homing Chemokines,” J. Leukoc. Biol. 69:331-339 (2001)). Indeed, ectopic expression of transgenic lymphotoxin or BLC chemokine is sufficient to induce lymphoid neogenesis (Kratz et al., “Chronic Inflammation Caused by Lymphotoxin is Lymphoid Neogenesis,” J. Exp. Med. 183:1461-1472 (1996); Luther et al., “BLC Expression in Pancreatic Islets Causes B Cell Recruitment and Lymphotoxin-Dependent Lymphoid Neogenesis,” Immunity 12:471-481 (2000); Drayton et al., “Ectopic LTαβ Directs Lymphoid Organ Neogenesis with Concomitant Expression of Peripheral Node Addressin and a HEV-restricted Sulfotransferase,” J. Exp. Med. 197:1153-1163 (2003)).

B cell activation and terminal differentiation during a humoral immune response represent the integrated result of the temporal, spatial, and kinetic modulation of signals from a number of surface receptors involved in antigen recognition (the B cell receptor), cell-cell interactions (such as CD40, OX40 ligand, adhesion receptors), and detection of soluble lymphokines (reviews in Klaus, “B Cell Activation,” In Molecular Immunology, Second edition, ed. Hames et al., pp. 248-282. Oxford: Oxford University Press (1996); Fu et al., “Independent Signals Regulate Development of Primary and Secondary Follicle Structure in Spleen and Mesenteric Lymph Node,” Proc. Natl. Acad. Sci. USA 94:5739-5743 (1997); Parker, “T Cell-Dependent B Cell Activation,” Annu. Rev. Immunol. 11:331-360 (1993)).

The interpretation of all these signals by the B cell is largely dependent on the microenvironmental context in which they are delivered, as highlighted by the strict localization of the successive steps of the B cell activation process (Tarlinton D., “Germinal Centers: Form and Function,” Curr. Opin. Immunol. 10:245-251 (1998); MacLennan I. C., “Germinal Centers,” Annu. Rev. Immunol. 12:117-139 (1994)). Thus, during a classical T cell-dependent response, follicular B cells usually encounter antigen within the PALS, in the course of their migration from afferent vases to the B cell follicles. Here, interactions can form between antigen-specific B, T cells and DCs, resulting in a first surge of lymphocyte activation, initial expansion of antigen-specific clones, and the initiation of a primary immune response, which provides a rapid burst in antibody production via the generation of resident, short-lived plasma cells. Lymphocyte clones generated in this first phase leave the PALS and proceed to the follicles, where they initiate the formation of GCs. Within GCs, highly proliferating B cells (centroblasts) expand the pool of antigen-specific cells and undergo somatic hypermutation of their Ig genes, which changes the affinity of the antibody for its antigen. B cells emerging from this phase undergo selection for their affinity to antigen, present in immune complexes on the surface of FDCs. High-affinity clones receive surface Ig-mediated and survival signals, which allow them to further differentiate, after interaction with TH cells, into plasmablasts and memory B cells. These exit the GC and leave the lymphoid organ microenvironment as circulating memory B cells, or as long-lived plasma cells, which usually home back to the bone marrow for long-term antibody production.

Responses to many bacterial, polysaccharide, and highly repetitive antigens do not require T cell help and do not lead to germinal center formation (Mond et al., “T Cell Independent Antigens,” Curr. Opin. Immunol. 7:349-354 (1995); Mond et al., “T Cell-Independent Antigens Type 2,” Annu. Rev. Immunol. 13:655-692 (1995); Fagarasan et al., “T-Independent Immune Response: New Aspects of B Cell Biology,” Science 290:89-92 (2000); Martin et al., “B-Cell Subsets and the Mature Preimmune Repertoire. Marginal Zone and B 1 B Cells as Part of a “Natural Immune Memory,” Immunol. Rev. 175:70-79 (2000); Martin et al., “B1 Cells: Similarities and Differences With Other B Cell Subsets,” Curr. Opin. Immunol. 13:195-201 (2001)). Even in this case, however, the specific spatial and temporal organization and interaction of individual cell subsets (most notably, MZB and B-1 cells) within restricted anatomical compartments (such as the MZ) is necessary for the development of an immune response (Fagarasan et al., “T-Independent Immune Response: New Aspects of B Cell Biology,” Science 290:89-92 (2000); Martin et al., “B-Cell Subsets and the Mature Preimmune Repertoire. Marginal Zone and B1 B Cells as Part of a “Natural Immune Memory,” Immunol. Rev. 175:70-79 (2000); Martin et al., “B1 Cells: Similarities and Differences With Other B Cell Subsets,” Curr. Opin. Immunol. 13:195-201 (2001); Vos et al., “B-Cell Activation by T-Cell-Independent Type 2 Antigens as an Integral Part of the Humoral Immune Response to Pathogenic Microorganisms,” Immunol. Rev. 176:154-170 (2000); Garcia de Vinuesa et al., “T-Independent Type 2 Antigens Induce B Cell Proliferation in Multiple Splenic Sites, but Exponential Growth is Confined to Extrafollicular Foci,” Eur. J. Immunol. 29:1314-1323 (1999); Martin et al., “Marginal Zone and B1 B Cells Unite in the Early Response Against T-Independent Blood-Borne Particulate Antigens,” Immunity 14:617-629 (2001)).

The delivery of an activation signal is required for survival of B cells in vitro. In the absence of such signals, >90% of follicular B cells undergo apoptosis within 72 hours of culture, a process that can be inhibited by transgenic expression of antiapoptotic genes, such as bcl-2 (McDonnell et al., “Bcl-2-Immunoglobulin Transgenic Mice Demonstrate Extended B Cell Survival and Follicular Lymphoproliferation,” Cell 57:79-88 (1989); and Strasser et al., “Enforced BCL2 Expression in B-Lymphoid Cells Prolongs Antibody Responses and Elicits Autoimmune Disease,” Proc. Natl. Acad. Sci. USA 88: 8661-8665 (1991)). Interestingly, rapid apoptosis of murine mature B cells following deletion of their Ig coding sequences in inducible Cre-Lox transgenic mice suggests that low-level, “tonic” signals from the BCR are also likely required for survival of peripheral B lymphocytes in vivo (Lam et al., “In vivo Ablation of Surface Immunoglobulin on Mature B Cells by Inducible Gene Targeting Results in Rapid Cell Death,” Cell 90:1073-1083 (1997)). B1 cells appear to have a somewhat different in vitro kinetics, surviving in greater numbers during the first few days of culture (about 50% at day 7), then undergoing a crisis from which isolated long-lived, clonal populations can arise after 6-8 weeks (Braun J., “Spontaneous In vitro Occurrence and Long-Term Culture of Murine B Lymphoblast Cell Lines,” J. Immunol. 130:2113-2116 (1983)). Such clones, although non-tumoral, appear to be able to propagate indefinitely in vitro and display some molecular hallmarks of transformed cells, including the amplification of c-myc (Braun J., “Spontaneous In vitro Occurrence and Long-Term Culture of Murine B Lymphoblast Cell Lines,” J. Immunol. 130:2113-2116 (1983); Citri et al., “Elevated myc Expression and c-myc Amplification in Spontaneously Occurring B Lymphoid Cell Lines,” J. Exp. Med. 165:1188-1194 (1987)). The long-term maintenance of resting B lymphocytes in the absence of over-expression of proto-oncogenes is therefore unfeasible using current technology.

Splenocytes from mice that have been previously antigen-primed (i.e., immunized) in vivo, but not naïve splenocytes, are able to mount relatively weak but detectable antigen-specific antibody responses in vitro (Marcelletti et al., “Elicitation of Antigen-Induced Primary and Secondary Murine IgE Antibody Responses In vitro,” Cell. Immunol. 135:471-489 (1991), and references therein). As discussed above, the absence of primary responses in these conditions may be at least in part due to the short life span of resting B lymphocytes in vitro. Thus, the more rapid entry into cell cycle of antigen-experienced vs. naïve lymphocytes upon antigen stimulation (Banchereau et al., “Molecular Control of B Lymphocyte Growth and Differentiation,” Stem Cells 12:278-288 (1994); Tough et al., “Stimulation of Naive and Memory T Cells by Cytokines,” Immunol. Rev. 170:39-47 (1999); Sprent, “Lifespans of Naive, Memory and Effector Lymphocytes,” Curr. Opin. Immunol. 5:433-438 (1993)) would allow the former, but not the latter, to become activated before apoptosis occurs. Furthermore, human activated, germinal center B cells can be made to differentiate into memory cells or plasma cells in vitro in the appropriate culture conditions (Arpin et al., “Generation of Memory B Cells and Plasma Cells In Vitro,” Science 268 (1995)). Altogether, these findings suggest that primary, ex vivo lymphocytes are competent to respond to specific signals and differentiate into effector and memory cells in vitro, but that the lymphoid organ microenvironment provides specific signals for primary immune response initiation, cell survival and differentiation.

Blood cell formation in normal adults takes place in the extravascular space between bone marrow sinuses. Besides the hematopoietic cells, the marrow also contains stromal cells including the endothelial cells, reticular cells, and macrophages. The stromal cells, their cell processes, and the extracellular matrices they secrete form a three-dimensional scaffolding upon which the hematopoietic cells lodge. The stromal cells, through their intimate physical contact with the hematopoietic cells, the extracellular matrices (ECM) and the growth factors they secrete, create the intricate “Hematopoietic Inductive Microenvironment” (HIM), which regulates the proliferation and differentiation of the hematopoietic cells (Abboud et al., “Structure of the Bone Marrow,” In Williams Hematology, Beutler et al., eds., New York: McGraw-Hill, pp. 25-28 (1995)). It has been hypothesized that “niches”, formed by the stromal cells, direct the hematopoietic cells towards self-renewal or differentiation into specific lineages.

Most studies of in vitro hematopoiesis have used the murine long-term bone marrow culture (LTBMC) system, first developed by Dexter and co-workers (Dexter et al., “Proliferation of Haemopoietic Stem Cells In Vitro,” Br. J. Haematol. 28:525-530 (1974)) employing tissue culture flasks or bottles. In the Dexter culture, the stromal cells spread and attach to the surface of the culture flask, forming a flat adherent layer. The stromal cells become extremely flattened and are therefore called “blanket cells.” The hematopoietic cells loosely bind to the stromal layer (the adherent compartment) where they proliferate and differentiate. The mature blood cells and some of the progenitor cells are released into the culture medium (the non-adherent compartment). Active growth of hematopoietic cells results in hematopoietic foci with clusters of cells, described as “the cobblestone areas” due to their distinct appearance under a phase-contrast inverted microscope. Under these conditions, normal B-lymphoid cells in 2D bone marrow cultures become progressively skewed toward precursor B-cell populations that do not retain a normal immunophenotype, and mature B-lymphocytes, such as those harvested from spleen or lymph node, do not survive beyond several days ex-vivo in the absence of mitogenic stimulation.

Despite its ability to support hematopoietic differentiation, several limitations are obvious in the Dexter culture system. Most notably, the mature cells produced in the “classic” cultures are mainly neutrophils and monocytes/macrophages (especially in the murine system), and specific alterations have to be introduced to drive differentiation of other cell lineages (Hocking et al., “Long-Term Human Bone Marrow Cultures,” Blood 56:118-124 (1980); Slovick et al., “Survival of Granulocytic Progenitors in the Nonadherent and Adherent Compartments of Human Long-Term Marrow Cultures,” Exp. Hematol. 12:327-338 (1984)). In addition, hematopoiesis in the flask culture is accompanied by extensive lipogenesis of the stromal cells and formation of fat cells, which are rarely found in normal human marrow with active hematopoiesis and are virtually absent in the marrow of mouse femur. In addition, the optimal temperature for cell output and duration of Dexter culture is 33° C., a temperature not optimal for growth of all cells.

Peripheral lymphoid organs represent critical sites for the development of adaptive immune responses. They are characterized by a complex histological organization in which specific localization of cell subsets, their physical interactions and migration during the course of the immune response are tightly controlled by microenvironmental signals such as cytokines, chemokines, adhesion molecules and homing receptors. The essential role these factors play in immune system function has hampered the faithful replication of normal immune mechanisms in vitro. Indeed, even the long-term culture of peripheral lymphocytes in the absence of activating stimuli or cell transformation has not been achieved so far.

The present invention is directed to overcoming these and other deficiencies in the art.

### SUMMARY OF THE INVENTION

The present invention relates to a method of culturing peripheral lymphoid organ cells. This method involves culturing peripheral lymphoid organ cells on a three-dimensional scaffolding which is covered or surrounded with culture medium under conditions effective to generate and maintain mature and functional peripheral lymphoid organ cells. The three-dimensional scaffolding allows cells in the culture medium to have cell to cell contact in three dimensions.

The present invention also relates to a method of screening for vaccine candidates for efficacy in eliciting an immune response. This method involves culturing peripheral lymphoid organ cells in a container on a three-dimensional scaffolding which is covered or surrounded with culture medium under conditions effective to generate and maintain mature and functional peripheral lymphoid organ cells. The three-dimensional scaffolding allows the peripheral lymphoid organ cells in the culture medium to have cell to cell contact in three dimensions. A vaccine candidate is added to the container, and it is determined whether the vaccine candidate elicits an immune response in cultured peripheral lymphoid organ cells.

Another aspect of the present invention is a method of identifying genes or proteins which are related to peripheral lymphoid organ cell formation or function. This method involves culturing peripheral lymphoid organ cells on a three-dimensional scaffolding which is covered or surrounded with culture medium under conditions effective to generate and maintain mature and functional peripheral lymphoid organ cells. The three-dimensional scaffolding allows the cells in the culture medium to have cell to cell contact in three dimensions. One or more culture conditions in a test culture are altered, and peripheral lymphoid organ cell number and function in the test culture are determined. Screening for genes or proteins associated with a change in peripheral lymphoid organ cell number or function in the test culture is carried out.

The present invention also relates to a method of screening for drugs effecting peripheral lymphoid organ cell generation, maturation or function. This method involves culturing peripheral lymphoid organ cells in a container on a three-dimensional scaffolding which is covered or surrounded with culture medium under conditions effective to generate and maintain mature and functional peripheral lymphoid organ cells. The three-dimensional scaffolding allows the cells in the culture medium to have cell to cell contact in three dimensions. A test compound is added to the container, cultured cells are removed from the container, and the test compound's ability to effect peripheral lymphoid organ cell generation, maturation or function is determined.

Another aspect of the present invention is a method of treating a patient for a disease condition. This method involves culturing peripheral lymphoid organ cells on a three-dimensional scaffolding which is covered or surrounded with culture medium under conditions effective to generate and maintain mature and functional peripheral lymphoid organ cells. The three-dimensional scaffolding allows the cells in the culture medium to have cell to cell contact in three dimensions. An effective amount of immune system cells produced in the three dimensional cell culture system is administered to the patient, thereby treating the patient for the disease condition.

The present invention also relates to a method for effecting gene expression of peripheral lymphoid organ cells. This method involves culturing peripheral lymphoid organ cells on a three-dimensional scaffolding which is covered or surrounded with culture medium under conditions effective to generate and maintain mature and functional peripheral lymphoid organ cells. The cultured peripheral lymphoid organ cells are transformed or transduced with a desired gene, thereby effecting the gene expression of the peripheral lymphoid organ cells.

The long-term in vitro maintenance of normal peripheral lymphocytes provided by the present invention represents a major advance for a number of basic immunological studies. Furthermore, culture of lymphocytes and other immune accessory cells in the appropriate microenvironment may allow replication of an antigen-specific immune response in an accessible, easily modifiable setting. Potential applications for lymphoid organ cell culture method of the present invention range from the testing of immunogenicity of vaccine candidates in vitro, to the generation of human monoclonal antibodies, to immunotherapy interventions. The present invention provides a culture system highly suited to these studies because of its physiologic relevance, its limited size, easy sampling, and increased access.

### BRIEF DESCRIPTION OF THE DRAWINGS

FIGS. 1A-C shows the structure of the splenic follicle. Adjacent sections from a normal C57B1/6 mouse spleen were stained with fluorescent-labeled antibodies to T cells (CD3ε, green, FIG. 1A) vs. B cells (B220, orange, FIG. 1A), and for follicular B cells (IgD, green, FIG. 1B) vs. marginal zone B cells (IgM, orange, FIG. 1B). FIG. 1C highlights the relevant regions: the T cell zone (TZ), or PALS, the B cell follicle (BF) and the marginal zone (Mz).

FIGS. 2A-B are scanning electron micrographs showing the structural organization of murine bone marrow cells growing inside the pores of a collagen microsphere 3D bone marrow bioreactor (week 2). FIG. 2A shows hemopoietic cells grown in clusters in a three-dimensional fashion. In FIG. 2B spherical leukocytes and biconcave red cells are clearly visible.

FIG. 3 is a schematic drawing of the 3D fed-batch bioreactor of the present invention. The figure is not drawn to scale.

FIGS. 4A-F show B220 and IgM cell surface expression in long-term cultures of B lymphocytes in 3D bioreactors. Cell samples from a 3D splenic culture were taken at different time points (week 1-8, FIG. 4B-F, respectively, washed in PBS, 5% fetal calf serum (FCS) and stained for 15 min. on ice in the same buffer with fluorescent antibodies to the B cell antigens B220 and IgM, at 0.25 μg antibody for 0.1-0.5×10⁶ cells in 50 μl. After extensive washing, cells were analyzed by FACS on a Becton Dickinson FACScalibur flow cytometer. FACS plots (B220-vertical axis, IgM—horizontal axis) of live-gated cells, generated with CellQuest software (BD), are shown, together with a representative fresh spleen sample (FIG. 4A). IgM, B220-positive cells persist as late as week 8 of culture. Note the slightly lower IgM levels in cultured B cells.

FIGS. 5A-H show B 1 and B2 marker expression on 3D culture B cells. B cells from a 2 week-old 3D culture, (FIGS. 5E-F) as well as from fresh spleen (FIGS. 5A-B) and peritoneal cavity (PC) lavage (FIGS. 5C-D), were stained with antibodies to B220 (vertical axis) and the surface marker CD23 (present on B2 cells), or CD5 (present on B1a and T cells) (FIGS. 5A, 5C, 5E, respectively). The two panels on the right (FIGS. 5G-H) show the relative position of B1, B2 B cells, T cells (T) and other cells (O) within the plots. 3D culture B cells display markers for both B2 cells (CD23) and B1a cells (CD5).

FIGS. 6A-B are comparisons of cells from a week 4 spleen culture and fresh splenocytes stained for IgM, B220, and IgD expression, and analyzed by FACS. 3D B cells are similar in size to small, resting B cells, and express IgD. FIG. 6A shows the forward scatter profile (which corresponds to cell size) of live-gated IgM, B220-positive cells from the two samples. Note that the 3D B cells peak (thick line) overlaps with that of the small resting B cell population in the spleen sample (thin line). FIG. 6B shows the IgD profile in the same cells: 3D B cells express significant levels of IgM, although somewhat (about 3-fold) lower than the average spleen B cell.

FIGS. 7A-D are a comparison of 3D spleen cultures and fresh splenocytes stained for T-cell markers. Fresh spleen samples (FIGS. 7A-B), as well as samples from 3D cultures week 1 (FIG. 7C) and week 3 (FIG. 7D), as were stained for expression of the CD4 (vertical axis) and CD8 (horizontal axis) T cell markers, characteristic of the helper and cytotoxic T cell subsets, respectively. Abundant T cells of either type are found in the cultures at both time points, indicating that 3D spleen cultures harbor T lymphocytes.

FIGS. 8A-H are a comparison of peripheral lymph node cultures with fresh abdominal lymph node cells. 3D cultures were established from peripheral mouse lymph nodes (pooled axillary, inguinal, and abdominal). Cells from fresh abdominal lymph nodes (FIGS. 8A, 8C, E, and 8G) and 3D cells at week 2 (FIGS. 8B and 8F) and 5 of culture (FIGS. 8D and 8H) were stained with antibodies to B220 and IgM (B cells, and CD3ε(T cells) and analyzed by FACS. Presence of B220, IgM-positive B cells (FIGS. 8A-D) and CD3-positive T cells (FIGS. 8E-H), is clearly detected at both time points, indicating that 3D peripheral lymph node cultures harbor B and T lymphocytes.

FIGS. 9A-L show the in vitro activation of 3D cultured spleen cells. Fresh splenocytes and cells from a 2 week old 3D spleen culture were placed in culture at 10⁶ cells/ml in the presence of bacterial lipolysaccharide (LPS, a polyclonal activator of B cells) at 20 μg/ml. Cultures were maintained for 5 days, feeding the cells daily from day 2 onward. At day 5, cells were collected, washed and stained with antibodies to IgG2b (FIGS. 9A-B) and IgG3 (FIGS. 9C-D) (two IgG subclasses whose expression is induced by LPS stimulation); Syndecan-1/CD 138 (FIGS. 9E-F), a marker of Ig-secreting plasmacytoid cells; CD23 (FIGS. 9G-H); CD80 (FIGS. 9I-J), and CD86 (FIGS. 9K-L), whose expression is modulated by LPS stimulation. FACS analysis was performed as in FIG. 1, with the difference that specific settings were used to collect events from large, activated cells. A fraction of 3D cultured B cells switched to IgG2b (FIG. 9B, boxed), and IgG3 expression (FIG. 9D, boxed), and expressed the CD138 marker (FIG. 9F, boxed), with a pattern similar to normal cells (FIGS. 9A, 9C, and 9E, respectively). Down-regulation of CD23 (FIG. 9H), up-regulation of CD80 (FIG. 9J) and CD86 (FIG. 9L) in LPS-activated 3D cells also paralleled analogous changes in normal cell cultures (FIGS. 9G, 91, and 9K, respectively.). While some variations are observed (slightly higher IgG switching and CD80 expression, and somewhat lower CD138 and CD86 expression in 3D cells), these results indicate that 3D cells are fully competent to respond to activation signals.

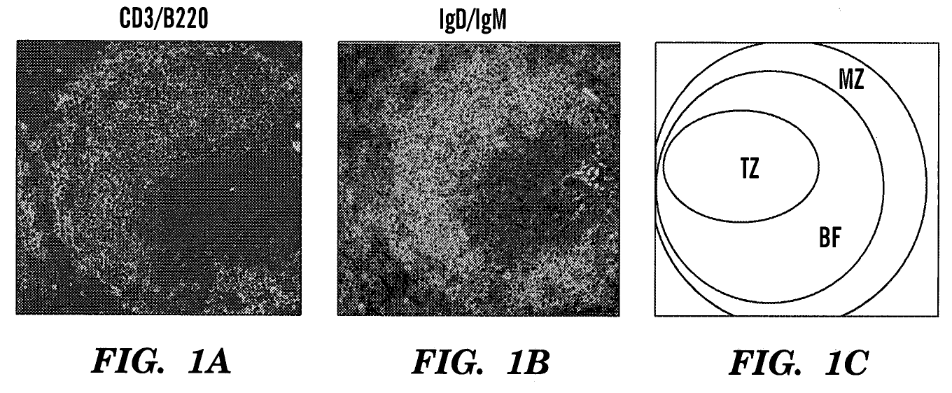

### DETAILED DESCRIPTION OF THE INVENTION

The present invention relates to a method of culturing peripheral lymphoid organ cells. This method involves culturing peripheral lymphoid organ cells on a three-dimensional scaffolding which is covered or surrounded with culture medium under conditions effective to generate and maintain mature and functional peripheral lymphoid organ cells, where the three-dimensional scaffolding allows cells in the culture medium to have cell to cell contact in three dimensions.

The 3-D culture system of the present invention includes a chamber or container having a scaffolding covered or surrounded in culture medium where the scaffolding extends vertically, horizontally, and depthwise. This allows for the lymphoid organ cells to have cell to cell contacts in each of these three dimensions.

A “3D culture” as used herein is a cell culture method providing a structural support, for example, a porous scaffold, that allow cells to form three-dimensional clusters (cells growing in aggregates) by growing in or on all faces, or available surfaces of the support. The dimensions, i.e., the height, length, and width, of the support and cell clusters can be varied as needed.

A 3D culture system is important for successful long-term culturing of peripheral lymphoid organs is not just because of the use of extracellular matrix substrate (e.g., collagen), or the presence of non-specific feeders, but the very specialized and compartmentalized interactions between several cell types, including, at the very least, stroma, B and T lymphocytes, follicular dendritic cells, and dendritic cells that occur in the microenvironment. These cells do not just adhere to each other, but are involved in cell-cell communication through surface receptors, adhesion molecules, and soluble factors (chemokines, cytokines). Moreover, cells, especially B lymphocytes, move from one compartment to the other in the course of normal trafficking and during an immune response in a highly regulated fashion. In short, there is no way that the environment provided by the 3D bioreactor of the present invention can be created on a 2D system using, for example, a collagen layer or a layer of fibroblast feeders. As used herein, “generation” is meant to include growth (i.e., increase in cell number) and differentiation.

Peripheral lymphoid organ cells include T lymphocytes (T-cells) and B lymphocytes (B-cells), natural killer (NK) cells, dendritic and follicular dendritic cells, granulocytes, macrophages, and stromal cell subsets. T-cells include, without limitation, cytotoxic T-cells, helper T-cells and combinations thereof. B-cells include, without limitation, immature B cells, naïve B cells, memory B-cells, plasma cells, and combinations thereof.

In accordance with the present invention, a bioreactor system and method for generating lymphoid organ cells is provided. The bioreactor of the present invention provides a three-dimensional structure which mimics the natural extracellular matrix and ample surface area of the in vivo microenvironment and allows cell to cell interaction at a tissue-like cell density. It is understood that the bioreactor of the present invention may have many different configurations so long as it provides a three-dimensional structure. With respect to the bioreactor, the term “three-dimensional structure” is used interchangeably with the term “scaffolding”.

The bioreactor for use in generating lymphoid organ cells includes a container or vessel having at least one chamber or section with scaffolding located therein. The scaffold can be shaped into a heat valve, vessel (tubular), or any other suitable shape. Such constructs are well known in the art (U.S. Pat. No. 6,479,064 to Atala and U.S. Pat. No. 6,461,628 to Blanchard et al., which are hereby incorporated by reference in their entirety). The scaffolding is made of a porous or fibrous substrate, composed of a degradable, including bio-degradable substances, or a non-degradable material. Exemplary scaffolding materials include, without limitation, synthetic polymers, natural substances, semisynthetic materials, and any combination thereof. A culture media suitable for lymphoid organ cell culture is placed over or around the porous or fibrous substrate. Also suitable are scaffolding materials having slow-release formulas of growth factors.

FIG. 3 illustrates one possible configuration of a bioreactor which may be used to generate lymphoid organ cells. In FIG. 3, the porous or fibrous scaffolding is located in a lower culture chamber. It is understood that the bioreactor of the present invention may have any number of configurations so long as it provides a three dimensional structure (scaffolding).

Suitable material for the walls of the container or vessel include, without limitation, glass, ceramic, plastic, polycarbonate, vinyl, polyvinyl chloride (PVC), and metal. Culture medium which will support the growth and/or the maturation of lymphoid organ cells is placed over and/or around the porous or fibrous material.

Many different porous or fibrous materials may be used as scaffolding in the bioreactor such as, e.g., tangled fibers, porous particles, sponge, or sponge-like material. The porous or fibrous scaffolding allows lymphoid organ cells to lodge, proliferate, and differentiate. For purposes of example only and not limitation, suitable scaffolding substrates may be prepared using a wide variety of materials including natural polymers, such as polysaccharides and fibrous proteins, synthetic polymers, such as polyamides (nylon), polyesters, and polyurethanes, degradable polymers, such as PGA, PGLA, and minerals, including ceramics and metals, coral, gelatin, polyacrylamide, cotton, glass fiber, corrageenans, alginate, chitin, and dextrans. Examples of tangled fibers include glass wool, steel wool, and wire or fibrous mesh.

Examples of porous particles include, without limitation, beads, slabs, cubes, and cylinders (made from glass, plastic, or the like) cellulose, agar, hydroxyapatite, treated or untreated bone, collagen, gels such as Sephacryl, Sephadex, Sepharose, agarose or polyacrylamide. “Treated” bone may be subjected to different chemicals such as e.g., acid or alkali solutions. Such treatment alters the porosity of bone. If desired, the substrate may be coated with an extracellular matrix or matrices, such as, e.g., collagen, matrigel, fibronectin, heparin sulfate, hyaluronic and chondroitin sulfate, laminin, hemonectin, or proteoglycans.

The fibrous or porous material used as scaffolding in the bioreactor forms openings or pores into which lymphoid organ cells enter. Once entered, the cells become entrapped or adhered to the fibrous or porous material and colonize and/or aggregate thereon. Cell attachment and colonization can occur merely by inoculating the cells into the culture medium which overlays and/or surrounds the porous or fibrous substrate. Cell attachment and colonization may also occur by inoculating the cells directly onto the porous or fibrous substrates.

In accordance with the present invention, lymphoid organ cells must be able to enter the openings (pores) of the fibrous or porous material. The skilled artisan is cognizant of the different sizes of lymphoid organ cells and therefore the pore size needed to accommodate such cells. Generally speaking, a pore size in the range of from about 15 microns to about 1000 microns may be used. Preferably, a pore size in the range of from about 100 microns to about 300 microns is used.

In a preferred embodiment, a membrane is placed in the bioreactor in order to facilitate gas exchange. The membrane is gas permeable and may have a thickness in the range of from about 10 to about 100 μm. In a more preferred embodiment, the membrane has a thickness of about 50 μm. The membrane is placed over an opening in the bottom or side of the chamber or container. In order to prevent excessive leakage of media and cells from the bioreactor, a gasket may be placed around the opening and/or a solid plate placed under or alongside the opening and the assembly fastened.

The cell medium used in the bioreactor may be any of the widely known media used to support growth and/or differentiation of lymphoid organ cells, and in particular, growth and differentiation of lymphoid organ cells into immune system cells. For example, the following classical media may be used and supplemented, if desired, with vitamin and amino acid solutions, serum, and/or antibiotics: Fisher's medium (Gibco), Basal Media Eagle (BME), Dulbecco's Modified Eagle Media (D-MEM), Iscoves's Modified Dulbecco's Media, Minimum Essential Media (MEM), McCoy's 5A Media, and RPMI media.

An exemplary culture medium comprises RPMI, approximately 50 μg/ml penicillin, approximately 50 mg/ml streptomycin, approximately 0.2 mM L-glutamine, 50 μM beta-mercaptoethanol and 10% fetal bovine serum. The medium chamber may be continuously perfused if desired. The dissolved oxygen concentration and pH of the media may be controlled by well known methods. Suitable culture conditions for use in the 3D system of the present invention will be selected as appropriate for the source of the cell, e.g., mouse cells or human cells.

The bioreactor is inoculated with lymphoid organ cells by gently adding, for example, by pipetting, into the three-dimensional scaffolding portion of the bioreactor. Alternatively, the lymphoid organ cells may be added to the culture covering and/or surrounding the three dimensional scaffolding. Cells will settle or migrate into the porous or fibrous material making up the scaffolding. The number of cells added to the bioreactor depends on the total area of the three-dimensional scaffolding and volume of culture media.

Lymphoid organ cell cultures are meant to include, without limitation, spleen cells, lymph node cells, thymus cells, Peyer's patches cells, and combinations thereof. In a preferred embodiment, cultured cells are spleen cells. The lymphoid organ cells of the present invention may be derived or isolated from any mammal, including, without limitation, a human.

In one aspect of the present invention, cultured cells express one or more surface markers selected from the group consisting of CD5, CD23, CD69, CD25, MHC class I or II, CD80/86, CD138, CD38, CD27, CD8, CD4, CD3, CD45-RO, CD45-RA, and any combination thereof.

In an other aspect of the present invention, cultured cells fail to express one or more surface markers selected from the group consisting of CD5, CD23, CD69, CD25, MHC class I or 11, CD80/86, CD138, CD38, CD27, CD8, CD4, CD3, CD45-RO, CD45-RA, and any combination thereof.

For an exemplary bioreactor having a culture chamber of about 3/16″ height by about 5/16″ width by about 5/16″ length and packed with about 0.01 g of a porous or fibrous substrate, the number of mononuclear cells added to the bioreactor may be anywhere in the range of from about 10⁴ to 10⁹ cells. Preferably, 4-6×10⁶ cells may be used to inoculate the bioreactor. Using these guidelines, one skilled in the art would be able to adjust the number of cells used to inoculate the bioreactor depending on the total area of the three-dimensional scaffolding, volume of culture media, type of three-dimensional scaffolding, and source of lymphoid organ cells.

The culture may be fed every second day with the culture medium. Various other ingredients may be added to the culture media. Such media is herein termed “supplemented”. The media may contain exogenous growth factors, cytokines, lymphokines, hormones, chemokines, interleukins, mitogens, antigens or antigenic fragments thereof, extracellular matrices, or other biologically active molecules, or combinations thereof. For example, suitable cytokines include, without limitation, interleukin-2, interleukin-4, interleukin-6, interleukin-10, interleukin-7, interleukin-12, flt-3 Ligand, stem cell factor, thrombopoietin, CD40 ligand, BAC-1, L-BCGF, soluble interleukin 6R, and combinations thereof.

Media is supplemented using an amount of any cytokine known in the art to be suitable, for example, based on the manufacturer's recommended concentration for a given cell number or volume of media in a culture container. For example, interleukin-2 may be added in an approximate amount of about 1000 Upper ml. Interleukin-7 may be added in an approximate amount of about 2 ng/ml. The aforementioned amounts are exemplary and empirical. The skilled artisan may therefore vary the amounts according to the bioreactor setup i.e., size, volume, number and source of cells. In a preferred embodiment, the cultures are fed daily with unsupplemented medium and every second day with the supplemented medium.

In another aspect of the present invention, the lymphoid organ cells are cultured in media that has no external mitogens added.

The cell culture is allowed to grow anywhere from about a few days to several weeks. Treatment, including, but not limited to immunization, for example, in preparation of production of antigen-specific lymphocytes for cellular immunotherapy, are carried on the cultured cells when cell counts or other relevant cell culture conditions are appropriate for a given treatment.

The present invention thus provides a method for long-term maintenance of functional immune system cells which involves culturing lymphoid organ cells on a three dimensional support.

In one embodiment, the initial 3D culture of peripheral lymphoid organ cells is seeded or reseeded with a fresh population of cells. As used herein, “seeded” means to add cells from a source, or of a cell type, that was not previously present in the 3D culture system. “Reseeded” means to add additional cells from a source, or of a cell type, already present in the 3D culture. In this aspect, the 3D culture may be seeded or reseeded with peripheral lymphoid organ cells, peripheral lymphoid cells, primary lymphoid organ cells, stem cells, or combinations thereof. The peripheral lymphoid organ cells in this aspect of the present invention include, without limitation, spleen cells, lymph node cells, Peyer's patches cells, and combinations thereof.

Examples of immune system cells maintained by the methods of the present invention include T lymphocytes, B lymphocytes, antigen presenting cells, natural killer cells, naïve cells, activated cells, memory cells, and progenitors or precursors thereof.

Examples of T lymphocytes which may be generated and maintained by the methods of the present invention include, for example, CD4⁺ and CD8⁺ cells.

Examples of B lymphocytes which may be generated and maintained by the methods of the present invention include, for example, CD19⁺, IgD⁺, CD23⁺ (follicular B2 cells); IgM^{hi}, CD23⁻ (B 1 cells); IgM^{hi}, CD23⁺, CD21⁺ (marginal zone B cells), CD19+, PNA+, GL-7+(germinal center cells and plasma cells).

In addition to the cell types mentioned above, human B cell subsets represented in the 3D cultures described by the present invention should include, for example, CD38−, IgD+naïve cells, IgD+, CD38+naïve-germinal center transitional, CD38+, IgD-germinal center cells, and IgD−, CD38-memory B cells.

The present invention also provides a method of immune cell maturation, selection, antigen presentation, or expansion. The method comprises removing the immune cells from the three dimensional bioreactor and inoculating a further, or additional, culture with the removed immune cells. Matured, expanded, and/or antigen-presenting cells (APCs) may be removed and selected from the further cell culture using well known methods as well as methods described herein. As used herein, “further cell culture” may include a three dimensional support (scaffolding), media which will support the growth of, or differentiation of lymphoid organ cells into immune system cells; i.e., a second three dimensional bioreactor.

Preferably, “further cell culture” is meant to include at least one of a spleen cell culture, a thymus cell culture, a lymph node cell culture, or liver cell culture system. Methods of culturing adult or fetal spleen cells, thymus cells, lymph node cells or liver cells are well known in the art.

The present invention also provides a method of B cell maturation, selection, antigen-presentation or expansion which comprises inoculating a further culture with antibody-producing B cells cultured in the three dimensional bioreactor of the present invention. The antibody producing B cells are produced by culturing lymphoid organ cells on a three dimensional support, allowing for the growth of, or differentiation into, effector immune system cells, immunizing the culture with an antigen or antigenic fragment thereof, and identifying the antibodies produced and isolating the B cells producing the antigen specific antibodies.

In yet another aspect of the invention, there is provided a method of T cell maturation, selection, and activation. The method comprises inoculating a further cell culture with antigen specific T cells. The antigen specific T cells are produced by culturing lymphoid organ cells on a three dimensional support, allowing for the growth of, or differentiation into, immune system cells, immunizing the culture with an antigen or antigenic fragment thereof, and isolating the T cells produced by the culture which are antigen specific.

The present invention further provides a method of natural killer cell maturation, selection, antigen presentation, or expansion. The method comprises removing immune system cells from the three dimensional bioreactor, isolating natural killer cells, and inoculating a further cell culture with the natural killer cells.

Immune cells may be harvested in any number of well known methods. The chamber may be treated with any suitable agent, such as collagenase, to release the adhering cells. Non-adhering cells may be collected as they are released into the medium. Cells may also be removed from the substrate by physical means such as shaking, agitation, etc. Thereafter, the cells are collected using any procedure known in the art such as for example, by pipetting or centrifugation. Preferably, non-adherent cells are released by gentle stirring and mixing the bed of porous or fibrous material and collected by centrifugation or sedimentation.

If desired, the cell samples collected from the bioreactor may be further enriched for immune system cells using well known methods of positive selection. Thus, for example, a solid support (such as beads) having an antibody that binds lymphoid organ cells conjugated thereto, may be mixed with the cell sample. In this way the lymphoid organ cell types may be isolated together or separately. If a mixed population of lymphocytes is desired, then the solid support should be conjugating to antibodies for all subtypes. If a particular subtype is desired, then a solid support having an antibody conjugated thereto which binds a particular lymphocyte may be used. Examples of antibodies which may be conjugated to a solid support include anti-CD4 (for helper T-cells), anti-CD8 (for cytotoxic T-cells), anti-CD19+(for B-cells), anti-IgD (for naïve mature B-cells), and anti-surface IgG (for antigen stimulated B-cells). Antibody conjugated beads with immune system cells bound thereto are then collected by gravity or other means such as a magnet, in the case of magnetic beads.

Negative selection may also be used as a means of enriching the immune system cell population and subpopulations, e.g., B-cells, T-cells, and NK-cells in the cell sample removed from the bioreactor. With a negative selection scheme, a solid support (such as beads) having conjugated thereto one or more antibodies which react with cells other than immune system cells, may be mixed with the cell sample. Antibody conjugated beads with cells other than immune system cells bound thereto are then collected by gravity or other means such as a magnet, in the case of magnetic beads.

Lymphoid-derived immune system cells may be identified using any well known method, including, without limitation, flow-cytometry analysis (FACS), immunocytochemistry, enzyme-linked immunospot (ELISPOT) (Coligan et al., In Current Protocols in Immunology, R. Coico, ed. John Wiley & Sons, Inc., New York (1994), which is hereby incorporated by reference in its entirety), and cytotoxicity assays for NK cells. These methodologies are well known in the art.

The cultured immune system cells of the present invention have a myriad of uses in therapeutic, diagnostic, and clinical settings. For example, the subject immune system cells may be used to produce antigen specific antibodies. Thus, in accordance with the present invention, there is provided a method for producing antigen specific antibodies. The method involved culturing lymphoid organ cells on a three dimensional support for a time and under conditions sufficient for the growth of, and/or differentiation into immune cells, immunizing the culture with an antigen or antigenic fragment thereof, and identifying antibodies produced by the culture system which are antigen-specific. The antigen or antigenic fragment can include, for example, peptide, protein, carbohydrate, glycoprotein, proteoglycan, lipopolysaccharide, nucleic acid, virus, whole bacteria, killed or lysed bacteria, cells, cell fragment, tissue, and combinations thereof. The virus, tissue mass, cell, or cell fragment may be live or dead. Any substance which can induce antibody production may be used. In one aspect of the present invention, the antigen is a tumor antigen. If desired, the antigen or antigenic fragment thereof may be combined with antigen presenting cells. In addition, the antigen or antigenic fragment may be presented as a conjugate. Examples of conjugates include diphtheria and tetanus oxoids. Immunization may be carried out with an adjuvant such as Freund's, muramyl dipeptide, or antigen-loaded dendritic cells.

Methods of immunizing cells are well known in the art and are described for example, in Fundamental Immunology 4th Ed., W. E. Paul, ed., Raven Press, New York, (1998), which is hereby incorporated by reference in its entirety. Methods of identifying antibodies which are antigen specific are well known and include, for example, ELISA, ELISPOT, flow cytometry, immunochemistry, and PCR.

Also in accord with the present invention, there are provided antibodies produced by the methods described hereinabove. Monoclonal antibodies are usually produced using well known methods such as those originally described by Milstein and Kohler, Nature 256:495-497 (1975), which is hereby incorporated by reference in its entirety. In the prior art procedures, a mouse or suitable animal is injected with an antigen or a fragment thereof. The animal is subsequently sacrificed and spleen cells are fused with myeloma cells to produce a hybridoma. In one aspect of the present invention, the antibody producing B-cells, prepared as described herein above, are removed from the bioreactor and screened to isolate individual cells which secrete a single antibody species to the antigen. The antibody-producing B-cell is then immortalized by fusion with a myeloma cell, thereby producing an immortal, antigen-specific antibody-producing B cell line. Fusion with mammalian myeloma cells or other fusion partners capable of replicating indefinitely in cell culture is effected by standard and well-known techniques, for example, by using polyethylene glycol (“PEG”) or other fusing agents (Milstein and Kohler, Eur. J. Immunol., 6:511 (1976), which is hereby incorporated by reference in its entirety). In this way, B cell lines may then be derived which secrete the desired monoclonal antibody.

B cells and B cell lines which produce the subject antibodies may be isolated using well known methods such as those described in Fundamental Immunology, Raven Press, New York, W. E. Paul, 4th ed., (1998) which is hereby incorporated by reference in its entirety.

The present invention also provides a method for producing antigen specific T cells. The method comprises the steps of culturing lymphoid organ cells on a three dimensional support and allowing for growth of, or differentiation into, immune system cells; immunizing the culture with an antigen or antigenic fragment thereof, and identifying T cells produced by the culture which are antigen specific. T cells may be identified using well known methods in the art, such as immunocytochemistry for T cell receptors. For example, using immunocytochemistry for expression of CD4+, CD8+, αβ, or γδ, T cells may be identified.

An antigen or antigenic fragment used to immunize the culture in a method for producing antigen specific T cells may be a peptidoglycan, protein, glycoprotein, virus, tissue mass, cell, or cell fragment. The virus, tissue mass, cell, or cell fragment may be live or dead. The antigen may also be a viral antigen or a tumor antigen. In addition, the antigen or antigenic fragment may be presented as a conjugate. Examples of conjugates include diphtheria and tetanus oxoids. Immunization may be carried out with an adjuvant such as Freund's, muramyl dipeptide, or antigen-loaded dendritic cells.

The present invention also includes immortalized T-cell populations. In one aspect of the present invention, the immortalized T-cells are T-cell hybridomas. T-cell hybridomas can be produced by fusing antigen-primed T cells, prepared as described above, with cancerous T cells (thymoma cells) in a manner similar to the B cell-myeloma fusion described above. T-cell hybridomas do not secrete antibody, but possess other immunological functions, for example, secretion of lymphokines and expression of T-cell receptors with specificity for antigen-MHC. Antigen-specific T-helper, suppressor, and cytotoxic hybridomas have all been cloned (Kuby, J., Immunology, Chap. 7, W.H. Freeman & Co., New York (1992), which is hereby incorporated by reference in its entirety).

In accordance with the present invention, there is also provided a method for producing dendritic cells. The method comprises culturing lymphoid organ cells on a three dimensional support as described above, and allowing for the growth of, and/or differentiation into, dendritic cells.

Examples of dendritic cells which may be produced in accordance with the present invention include for example, dendritic cells from myeloid-committed precursors and dendritic cells from lymphoid-committed precursors.

If desired, after culturing the lymphoid organ cells on the three dimensional support and allowing for the growth of, and/or differentiation into dendritic cells, the dendritic cell population may be selectively enriched. Selective enhancement of dendritic cells may be performed by addition of a dendritic specific cytokine to the culture. Examples of dendritic specific cytokines include, interleukin-4, macrophage colony stimulating factor, stem cell factor, and fins-like tyrosine kinase 3 ligand.

The present invention therefore also provides dendritic cells produced by the method of culturing lymphoid organ cells on a three dimensional support and allowing for the growth of, and/or differentiation into, dendritic cells. Likewise, the present invention provides a dendritic cell line produced by a method of culturing lymphoid organ cells on a three dimensional support, allowing for the growth of, and/or differentiation into, dendritic cells and enhancing the production of a dendritic cell line by the addition of dendritic specific cytokine to the culture. Dendritic cells produced in accordance with the present invention may be isolated for example, by negative selection using immunomagnetic isolation methods.

The present invention provides a method of dendritic cell maturation, selection, antigen-charging, or expansion. The method comprises removing immune system cells cultured as described herein above from lymphoid organ cells, from the three dimensional bioreactor, isolating dendritic cells, and inoculating a further cell culture with the dendritic cells.

Another aspect of the present invention is a method of screening vaccines for efficacy in eliciting an immune response. This method involves culturing peripheral lymphoid organ cells in a container on a three-dimensional scaffolding which is covered or surrounded with culture medium under conditions effective to generate and maintain mature and functional peripheral lymphoid organ cells. The three-dimensional scaffolding allows the peripheral lymphoid organ cells in the culture medium to have cell to cell contact in three dimensions. A vaccine candidate is added to the container, and it is determined whether the vaccine candidate elicits an immune response in cultured peripheral lymphoid organ cells. Growing the peripheral lymphoid organ cells in a 3-D container of the present invention allows for the growth of, and/or differentiation of the lymphoid cells into effector immune system cells, i.e., T- or B- cells, as described above. As used herein, “vaccine” is meant to include any substance that induces an immune response, i.e., the activation of immune system cells. The type of immune response induced by the vaccine may be determined using well known methods such as ELISA, flow cytometry, and cytotoxicity assays. Cells and cell culture conditions suitable for the growth and/or differentiation of mature, functional lymphoid cells are all as described above.

Another aspect of the present invention is a method of identifying genes or proteins which are related to peripheral lymphoid organ cell formation or function. This method involves culturing peripheral lymphoid organ cells on a three-dimensional scaffolding which is covered or surrounded with culture medium under conditions effective to generate and maintain mature and functional peripheral lymphoid organ cells. The three-dimensional scaffolding allows the cells in the culture medium to have cell to cell contact in three dimensions. One or more culture conditions in a test culture are altered, and peripheral lymphoid organ cell number and function in the test culture are determined. Screening for genes or proteins associated with a change in peripheral lymphoid organ cell number or function in the test culture is carried out.

Examples of phenotypic changes which may be detected include for example, changes in surface marker expression and cytokine/chemokine expression. Such changes in phenotype may be detected using techniques such as flow cytometry, immunocytochemistry, and ELISPOT assay for antibody production cells.

Screening for the proteins or genes associated with a change in peripheral lymphoid cell number or function can be carried out using numerous procedures known in the art, including, without limitation, differential display, RNA arbitrarily primed (RAP)-PCR, and microarray analysis.

The present invention also relates to a method of screening for drugs effecting peripheral lymphoid organ cell generation, maturation, or function. This method involves culturing peripheral lymphoid organ cells in a container on a three-dimensional scaffolding which is covered or surrounded with culture medium under conditions effective to generate and maintain mature and functional peripheral lymphoid organ cells. The three-dimensional scaffolding allows the cells in the culture medium to have cell to cell contact in three dimensions. A test compound is added to the container, cultured cells are removed from the container, and the test compound's ability to effect lymphoid cell generation, maturation or function is determined. “To effect” as used herein refers to the ability of a test compound to inhibit, or alternatively, to stimulate, peripheral lymphoid cell maturation when added to a test culture of peripheral lymphoid cells. The determination of the ability to effect peripheral lymphoid cell maturation may be carried out using any procedures known in the art, including, without limitation, cell counting methods, immunohistochemistiy, flow cytometry, cytotoxicity, or a combination of these methods.

Expression of a gene in a lymphoid organ cell may be altered by any of the well known methods. For example, a lymphoid organ cell may be transfected or transduced with a genetic construct comprising a sequence which inserts itself into a gene. If the gene into which the sequence inserts itself is a gene involved in immune system cell development and function, the insertion of the foreign genetic sequence interrupts the gene and may manifest itself by a phenotypic change. Alternatively, an antisense molecule may be used to target a gene involved in immune system cell development and function. If treatment of a lymphoid organ cell with an antisense molecule results in a phenotypic change in the hemopoietic stem cell, then it may be deduced that the molecule targets a gene involved in immune system cell development and function. Naked DNA or RNA may also be used to transfect lymphoid organ cells. Alternatively, cells may be transduced, for example, by retroviral vectors.

There are many different methods of altering the expression of a gene in a lymphoid organ cell. Besides the gene interruption and antisense strategies described hereinabove, mutagenesis may also be used. For example, cells may be contacted or exposed to a mutagen, grown in the three dimensional support culture of the present invention, and then a determination made as to whether the mutagenized cells results in a phenotypic change in the cultured cells.

In one aspect of the present invention, the expression of the gene in the cultured cells may be compared to non-immune system cells or undifferentiated cells. Such a comparison has the purpose of examining their cellular function in relation to the gene of interest, and identifying any change in the cultured cells relative to non-immune or undifferentiated cell standard, or baseline.

In yet another aspect, after comparing the expression of the gene in the cultured cells to genes of cells in a non-immune producing culture, genes with altered expression between the first and second cultures are identified. In still another embodiment, the expression of the gene in cultured cells may be compared to cells having a different immune cell profile.

In accordance with the present invention, there are provided methods for determining the toxicity or efficacy of a drug. In this aspect of the invention, lymphoid organ cells are cultured on a three dimensional support and allowed to differentiate into immune system cells. A drug is administered to the cultured cells, and a determination is then made as to whether the drug is toxic to any of the cells in the culture. If the drug is either non-toxic or marginally toxic, a determination as to efficacy can then be made. As used herein, “drug” encompasses any element, molecule, chemical compound, hormone, growth factor, nucleotide sequence (including oligonucleotides), protein (including peptides), or reagents which have the ability to affect immune system cells. Thus for example, B cells may be affected in their ability to produce antibodies. T cells may be affected in their ability to mediate their cellular immunity functions, such as cytotoxicity. The present invention thus also provides immune system cells which have been exposed to a drug and which have survived such exposure.

In a typical toxicity or efficacy assay for a drug which affects immune system cells, cultured immune system cells are removed from the bioreactor and placed in a petri dish, flask, microscope slide, microtiter dish or the like, with enough culture medium or buffered solution to keep the cells alive. Cultured immune system cells may comprise mixed populations of cells, e.g., T cells, B cells, NK cells, and the like. Alternatively, subpopulations may be isolated and used in the toxicity assays. Preferably, a pH of approximately 7.2, and a temperature of about 37° C. is maintained. The number of immune system cells which may be used in a screening assay is empirical. Typically, a sample containing 1×10⁶ total cells may be used, depending upon the number of immune system cells in the cell sample.

The number of immune system cells in a cell sample relative to other cells may be determined microscopically by counting cells or immunohistochemically as described herein. Methods of cell counting are well known in the art. The concentration of the drug to be tested for toxicity or efficacy is empirical. One skilled in the art is familiar with methods of adjusting concentrations of different compositions in order to best identify the effects of a test compound in the screening assay. Typically, a range of concentrations is used and those portions of the range which exhibit serious deleterious effects on immune system cell viability are eliminated for further study. Those portions of the range having less deleterious effects on immune system cell viability are identified and used to further determine efficacy.

The mixture of immune system cells and drug is incubated for a time and under conditions sufficient for the inhibition or stimulation of immune function to be carried out. As defined herein, a sufficient time can be anywhere from about five minutes to several hours or more. When immune system cells are tested in a petri dish, flask, microscope slide, microtiter dish or the like, a sufficient time may be several minutes to several hours. Of course, the test time may be extended, if needed, in order to see effects on the cells. The skilled artisan is able to determine the optimal time for running the screening assay by removing samples and examining cells microscopically for viability.

A preferred buffer for use in the reactions is phenol red-free MEM supplemented with 1 X nonessential amino acids, 1X L-glutamine, 10% FBS, 50 U/ml penicillin, and 50 μg/ml streptomycin. In a preferred embodiment, the test reaction volume is between about 0.5 and about 2 ml. In a more preferred embodiment, the reaction volume is about 1 ml. In a preferred embodiment, the incubation temperature is approximately 37° C.

The test compound may be added to the culture medium or into the three dimensional scaffolding, thereby producing a “test culture.” The time at which the test compound is added is empirical but is relatively early in the culture period. Typically, control runs are performed in which no test compounds are added to the bioreactor.

Examples of drugs which may be tested for toxicity and efficacy by the methods of the present invention include for example, nucleic acids, modified nucleic acids, antibodies, chemotherapeutic agents, and cytokines. As described above, however, any available test compound may be used to screen for toxicity and/or efficacy on immune system cells. In some cases, the classification of a test compound as potential inhibitor or potential stimulator (inducer) of immune system cells is unknown and is initially determined by the assay.

Another aspect of the present invention is a method of treating a patient for a disease condition. This method involves culturing peripheral lymphoid organ cells on a three-dimensional scaffolding which is covered or surrounded with culture medium under conditions effective to generate and maintain mature and functional peripheral lymphoid organ cells. The three-dimensional scaffolding allows the cells in the culture medium to have cell to cell contact in three dimensions. An effective amount of immune system cells produced in the three dimensional cell culture system is administered to the patient, thereby treating the patient for the disease condition. Examples of such immune system cells suitable for cellular immunotherapy include T lymphocytes, B lymphocytes, antigen presenting cells, natural killer cells, naïve cells, activated cells, memory cells, and progenitors or precursors thereof. The aforementioned cells may be administered in any combination. If desired, only one of the aforementioned cell types may be administered. In one embodiment of this aspect of the present invention, the immune system cell is an antigen-specific lymphocyte, produced in the three dimensional bioreactor of the present invention as described herein above. By “effective amount” is meant an amount effective to treat the patient for a given disease condition or disorder. As used herein, “treating” is meant to include preventing or ameliorating a disease condition or disorder, including, without limitation, an immune disorder or condition of a patient. Thus, a patient susceptible to, or suffering from, any of the myriad of immune system conditions or disorders, cancer, or other disease condition, may be administered the subject immune system cells or progenitors or precursors thereof, in an amount effective to prevent or ameliorate the condition or disorder. Similarly, the surviving cells obtained from the subject drug toxicity or drug efficacy assays may be administered to a patient in an effective amount. This aspect is meant to encompass any mammals as “patients,” including, without limitation, mice, rats, and humans.

A patient may also be treated with an antigen specific antibody produced by the lymphocytes cultured in the 3D system of the present invention, as described, isolated, and prepared for administration using methods known in the art.

The present invention also relates to a method for effecting gene expression of peripheral lymphoid organ cells. This method involves culturing peripheral lymphoid organ cells on a three-dimensional scaffolding which is covered or surrounded with culture medium under conditions effective to generate and maintain mature and functional lymphoid cells. The three-dimensional scaffolding allows cells in the culture medium to have cell to cell contact in three dimensions. The cultured peripheral lymphoid organ cells are transformed or transduced with a desired gene, thereby effecting the gene expression of the peripheral lymphoid organ cells. The desired gene may be engineered in a vector to result in up- or down-regulation of expression of the gene. The gene may be homologous or heterologous to peripheral lymphoid organ cells. In this aspect of the present invention, a gene which is desired for the treatment of a disease condition is prepared in a suitable vector according to methods known in the art (Sambrook, et al., Molecular Cloning: A Laboratory Manual 3rd Ed., Cold Spring Harbor Laboratory Press (2001), which is hereby incorporated by reference in its entirety), and used to transform or transduce the cultured peripheral lymphoid organ cells of the present invention. Methods of transforming mammalian cells are known in the art, and may include the used of retroviruses. (See e.g., “Retrovirus Transformed Hemopoietic Progenitors” in Immunology Methods Manual, Academic Press, San Diego, I. Lefkovits, ed., (1997) which is hereby incorporated by reference in its entirety). Transformed or transduced lymphoid organ cells made in accordance with the method described herein are also provided. In an alternative embodiment, the culture contains helper cells which carry a vector containing the gene to be introduced.

The present invention also relates to a method of treating a patient for a disease condition. This method involves administering to a patient an effective amount of the transformed or transduced the cultured peripheral lymphoid organ cells prepared as described above. Administering may be carried out by any number of methods such as transplantation to a particular site in the body, such as a particular tissue or organ. In a preferred embodiment, the site is the spleen or a lymph node. Systemic infusion of cells may also be performed. In another embodiment, the gene may be targeted to immune system cells.

### EXAMPLES

### Example 1

### Three-D Culture System BioReactors

The 3D bioreactor system of the present invention was based on the hypothesis that lack of 3D structure and the consequent morphological distortion of the physiologic microenvironment could be responsible for the inability of long term in vitro cell cultures to support long-term maintenance of lymphoid cell cultures, as well as for the other alterations observed in the Dexter cultures (Wang et al., “Multilineal Hematopoiesis in a Three-Dimensional Murine Long-Term Bone Marrow Culture,” Exp. Hematol. 23:26-32 (1995); and Mantalaris et al., “Engineering a Human Bone Marrow Model: A Case Study on Ex vivo Erythropoiesis,” Biotech. Progr. 14:126-133 (1998)), which are hereby incorporated by reference in their entirety).

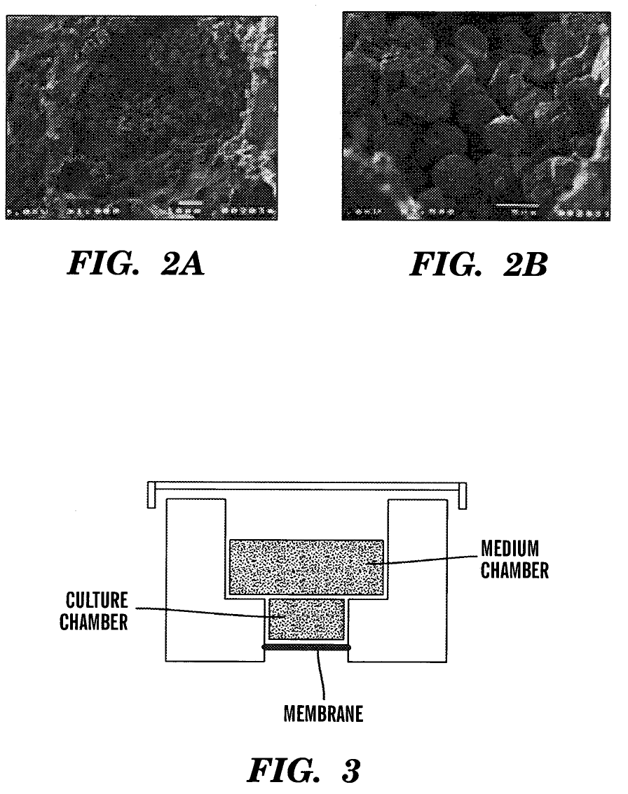

To test the feasibility of recapitulating the 3D structure and function of lymphoid organs in vitro, the scaffold-based, packed-bed bioreactor was adapted for culturing murine spleen cells using the principle previously demonstrated (Wang et al., “Multilineal Hematopoiesis in a Three-Dimensional Murine Long-Term Bone Marrow Culture,” Exp. Hematol. 23:26-32 (1995); and Mantalaris et al., “Engineering a Human Bone Marrow Model: A Case Study on Ex vivo Erythropoiesis,” Biotech. Progr. 14:126-133 (1998), and FIGS. 2A-B), which are hereby incorporated by reference in their entirety). The reactors and the porous scaffold for lymphoid cell culture are described below.

In one aspect of the present invention, the reactor is a fed batch bioreactor, as shown in FIG. 3. The fed batch reactor is fabricated using polycarbonate plates. The culture chamber ( 3/16″H× 5/16″W× 5/16″L) is packed with highly porous microcarriers (scaffolding) as described below. The packed-bed is overlayed with culture medium. The medium chamber (½″H× 5/16″W× 12/16″L) contains 0.6 ml of medium which is changed daily. A Teflon membrane is fabricated into the bottom of the culture chamber to facilitate gas exchange. The cells grow predominantly in the culture chamber, allowing daily or more frequent medium exchange in the medium chamber. Although a reactor of this particular design was used for the preliminary study in an exploratory manner for the proof-of-concept, the detailed design will be further optimized as the work progresses.

In addition to the fed-batch reactor, the present invention encompasses a previously described reactor with medium perfusion simulating the near steady-state environment of bone marrow (Wang et al., “Multilineal Hematopoiesis in a Three-Dimensional Murine Long-Term Bone Marrow Culture,” Exp. Hematol. 23:26-32 (1995), which is hereby incorporated by reference in its entirety). The perfusion design facilitates continuous medium supply, metabolic waste removal, and control of pH and dissolved oxygen levels. The center chamber of the reactor is packed with the scaffold material; the top and bottom chambers, separated from the center chamber by ultrafiltration membranes, form the medium compartment through which the culture medium is perfused. A peristaltic pump is used to recirculate the medium through the bioreactor and a reservoir.

The porous artificial scaffolds of the present invention provide a sponge-like, three-dimensional support and large surface area for cell attachment and population at a tissue-like density. Highly porous microbeads made of cellulose (1-2 mm diameter; 100-200 mm pore size; 95% porosity) were successfully used to support the peripheral lymphoid cultures described in the following sections. However, other materials, including degradable, bio-degradable, and non-degradable polymers, will be evaluated as part of a long-term study.

### Example 2

### Long-Term Culture of Unstimulated Splenic Lymphocytes

Total splenocyte preparations were generated by gently grinding fresh spleens from C57B1/6 mice in PBS, 5% fetal calf serum using frosted glass slides. About 20×10⁶ live leukocytes were seeded onto each bioreactor, and cultured in complete RPMI medium, 10% FCS. A small amount of cells was harvested weekly by gentle pipetting from the matrix bed, and analyzed for expression of B and T-cell specific surface markers.

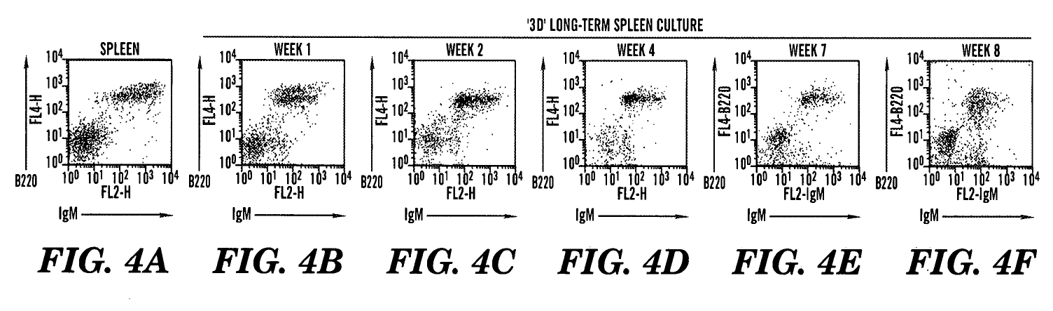

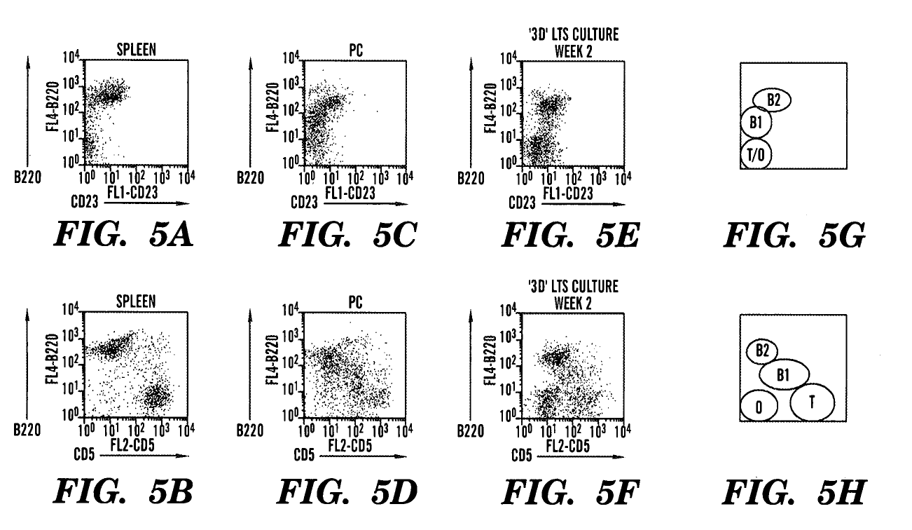

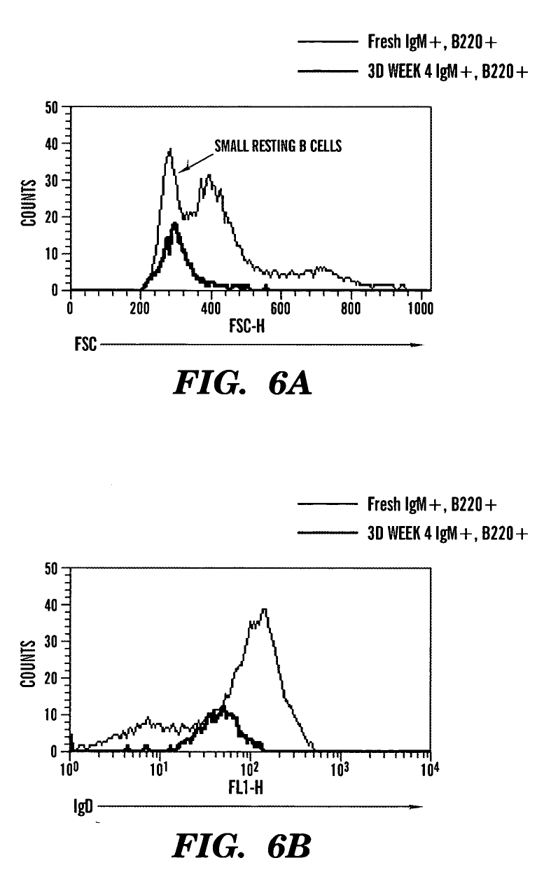

In striking contrast to flask cultures, significant fractions of B220/IgM positive B cells were routinely detected as far as 8 weeks into culture (when the cultures were terminated). As shown in FIGS. 4A-F, these B cells expressed somewhat lower B220 and IgM levels than the majority of B2 cells in the spleen. They also shared some features with peritoneal B 1 cells, most notably expression of low levels of the CD5 antigen, shown in FIGS. 5A-H. However, unlike B1 cells, 3D B cells expressed CD23, a defining marker of B2 B cells, shown in FIG. 5, and IgD, shown in FIG. 6, and were lacking CD11b expression. Note that the CD5 antigen can be induced on the surface of B2 cells by surface-Ig mediated stimuli (Cong et al., “Treatment of Murine CD5-B Cells with Anti-Ig, but not LPS, Induces Surface CD5: Two B-cell Activation Pathways,” Int. Immunol. 3:467-476 (1991), which is hereby incorporated by reference in its entirety). Thus, the presence of CD5 on 3D B cells may be either due to de novo expression on otherwise conventional B2-type cells, or to a preferential accumulation/survival of a subset of B 1a cells in the 3D culture system. Experiments aimed at defining the origin of these cells by selective transfer of sorted B 1 or B2 cells onto established cultures are underway.

Gated IgM/B220+B cells from 3D cultures were similar in size to the small, resting B cell population in the spleen, as assayed by forward light scatter on the flow cytometer. The gated IgM/B220+B cells from 3D cultures expressed lower but detectable IgD, which is normally rapidly down-regulated in activated B cells (Rabin et al., “Loss of CD23 is a Consequence of B-Cell Activation. Implications for the Analysis of B-Cell Lineages,” Ann. N.Y. Acad. Sci. 651:130-142 (1992), which is hereby incorporated by reference in its entirety), as shown in FIG. 6. In addition, no significant Ig secretion is observed in the culture supernatants after the first few weeks in culture, when splenic resident short lived plasma cells would still be present in the bioreactor. Altogether, these observations suggest that 3D B cells are not activated, although they do express low levels of the CD86 activation marker (about 100-fold less than in vitro activated B cells).

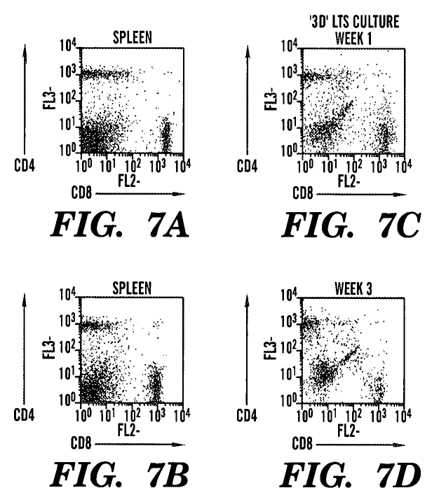

In addition to B lymphocytes, T cells of both CD4+ (T helper) and CD8+ (cytotoxic) lineages were also detected in the cultures, as shown in FIG. 7. A relative increase of the CD4+ vs. CD8+ ratio was observed, suggesting that the T helper subset may be preferentially generated and maintained in these culture conditions.

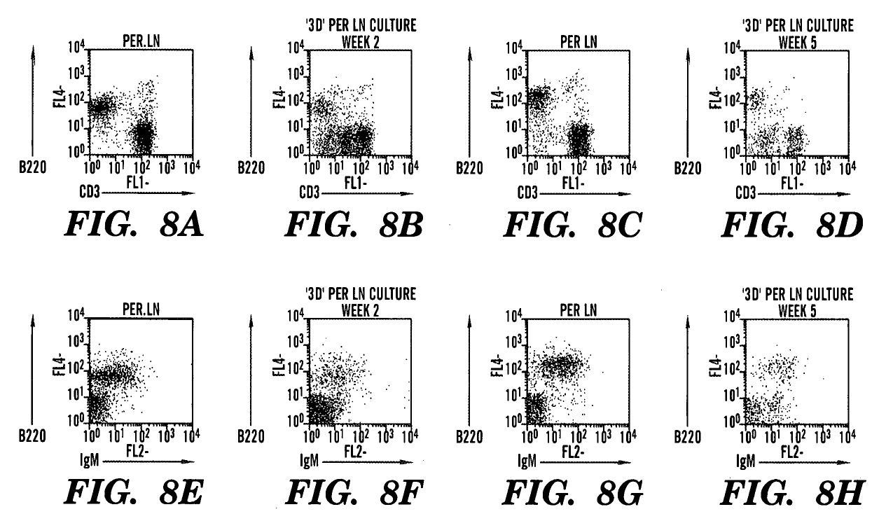

Finally, in a pilot experiment cells from pooled peripheral lymph nodes were seeded onto a 3D matrix. As shown in FIGS. 8B, 8D, 8F, 8H, lymph-node B and T cells also displayed extended survival in the 3D culture system.

### Example 3

### Long-Term 3D Culture B Cells Capable of Responding to in vitro Stimulation

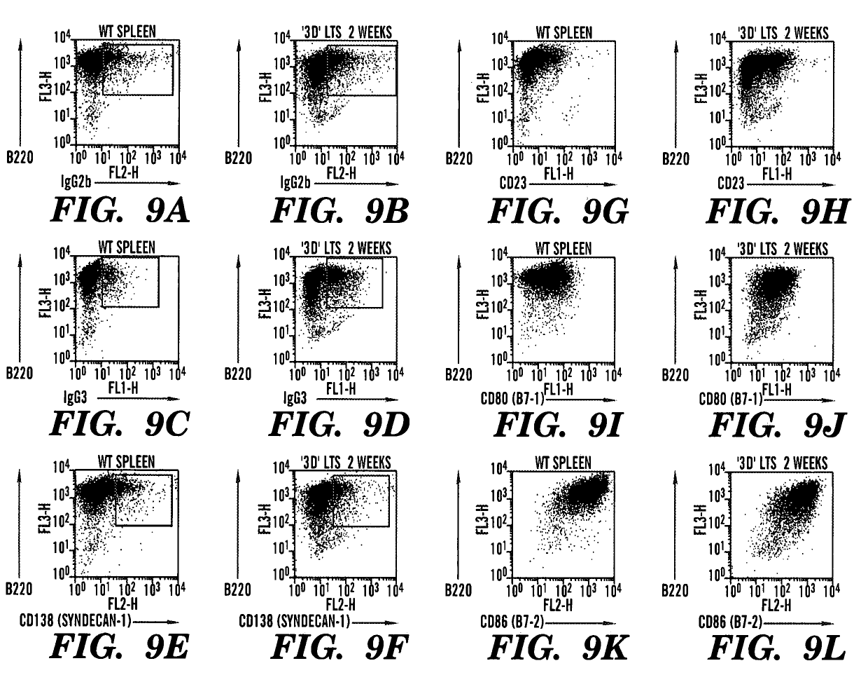

To test whether 3D culture B cells are functionally competent, B cells were stimulated from a 2-week culture with the polyclonal activator LPS, and their response to this stimulus was compared with that of normal, ex vivo-derived splenic B cells. As shown in FIG. 9, 3D B lymphocytes proliferated to an extent comparable to primary cells, and upregulated the co-stimulatory activation markers CD80 and CD86, as well as the marker Syndecan-1, characteristic of Ig-secreting plasma cells, to levels similar to controls. In addition, they were able to undergo immunoglobulin class switching to IgG, a highly specific gene rearrangement process unique to activated B cells (Bottaro et al., “Local and General Regulatory Elements of Immunoglobulin Class Switch Recombination,” In Molecular Mechanisms of IgE Regulation, Vercelli, ed., Chichester, England: J. Wiley and Sons, pp. 155-177 (1997), which is hereby incorporated by reference in its entirety).

Altogether, these data suggest that long-lived splenic B cells retain the ability to respond to appropriate stimuli, proliferate, and perform most of the critical functions associated with B cell activation. In summary, these data show that 3D bioreactors are able to support relatively long-lived populations of B and T lymphocytes, as well as other cells from peripheral lymphoid organs, in the absence of activation signals, to an extent not achievable by conventional culture techniques. These cells largely resemble, in phenotype and activation potential, their primary counterparts. These promising preliminary observations suggest that this novel culture method may be applied with success to the study of peripheral B lymphocyte biology, as well as to develop techniques and processes of biotechnological relevance.

Current culture systems for peripheral B lymphocytes rely on the effect of polyclonal activators, such as agonists of the antigen receptor (anti-Ig reagents), the CD40 surface protein (anti-CD40, or CD40 ligand in soluble or cell-bound form), or the Toll-like receptor family (bacterial lipopolysaccharide, LPS). In the absence of activators, the vast majority of B cells undergoes apoptosis within a few days, and only isolated quasi-transformed clones of a subset of B 1-type B cells can be established after prolonged culture (>6-8 weeks) (Braun J., “Spontaneous In vitro Occurrence and Long-Term Culture of Murine B Lymphoblast Cell Lines,” J. Immunol. 130:2113-2116 (1983)) which is hereby incorporated by reference in its entirety). Using an adaptation of a three-dimensional bioreactor system already successfully employed to replicate human and murine bone marrow hematopoiesis in vitro, unstimulated B and T lymphocytes from mouse lymph node and spleen have been successfully cultured as described herein for up to 8 weeks. These cells maintain phenotypes largely similar to their primary counterparts, and the ability to become activated in vitro upon appropriate stimulation. These findings make it possible, for the first time, to envision a system capable of closely recapitulating adaptive immune function in vitro. Fully implemented, this novel technology would have several remarkable applications. In basic immunological studies, it would allow the real-time analysis of human immune responses. Potential biotechnological uses include, among others, in vitro testing of vaccine candidates and immunomodulatory agents, and the generation of fully human (even entirely donor-matched) monoclonal antibodies.

Tissue engineering employing transplantable or extracorporeal bioreactors for culturing mammalian cells have started to show great promise in treating many hereditary or acquired organ or tissue disorders. These cells cultured in the bioreactors can be obtained from autologous, allogeneic, or xenogeneic sources to carry out specific therapeutic functions. Furthermore, they may be genetically engineered to carry a gene for replacing the defective one or augmenting the therapeutic effect of the cells. These bioartificial organs can be used for emergency or short-term clinical treatments such as in the case of the extracorporeal artificial liver. In other cases, they may be transplantable or implantable for long-term therapy. Cultured skin grafts and the encapsulated pancreatic islet cells are such examples.

Among all the tissues and organs that are potential targets of tissue engineering, the immune tissues/organs should be of prime importance since they constitute the body's defense system against infections and malignancies. However, developing tissue mimicry or model of the immune system, particularly its humoral arm involving B cell development and response, has been plagued by the technical challenges, which stem primarily from the need of a myriad of microenvironmental survival signals for maintaining B cells.

The results described herein indicate that, by providing 3D tissue-like structure, mature peripheral B and T cells can survive and remain competent in culture for several weeks. This provides an unprecedented opportunity for developing a lymph node model supporting long term B cell maintenance, activation and terminal differentiation. Such lymph node model will have significant ramifications in developing immune technologies ranging from ex vivo immune response, vaccine and drug testing, B cell adoptive transfer therapy, to human monoclonal antibody production as well as serve as a model for fundamental studies for delineating microenvironmental factors influencing B cell biology.

Furthermore, the present invention has significance as a 3D spleen model for studying peripheral lymphocytes. The ability to culture B lymphocytes in the absence of stimulation would represent a significant tool for immunological investigation, permitting the analysis of the homeostatic signals that contribute to lymphocyte survival, homing and interaction with their microenvironment. Furthermore, a culture system capable of supporting all the crucial immune cell types found in peripheral lymphoid organs (in particular, B and T lymphocytes, FDCs, DCs) in the proper ultrastructural context could be permitted to initiate, and possibly sustain, antigen-specific immune responses in an accessible setting amenable to a number of experimental manipulations. Such a system could also bypass the obvious complications associated with the study of human immune responses in vivo, and would allow a range of biotechnological applications, from the testing of immunogenicity of candidate vaccines in vitro, to the generation of human monoclonal antibodies, or immuno-therapeutic interventions.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow

1. A method of culturing peripheral lymphoid organ cells comprising:
culturing peripheral lymphoid organ cells on a three-dimensional scaffolding which is covered or surrounded with culture medium under conditions effective to generate and maintain mature and functional peripheral lymphoid organ cells, wherein said three-dimensional scaffolding allows cells in the culture medium to have cell to cell contact in three dimensions.

2. The method according to claim 1, wherein the scaffolding is selected from the group consisting of tangled fibers, porous particles, sponge, sponge-like material, and combinations thereof.

3. The method according to claim 1, wherein the scaffolding is formed from a material selected from the group consisting of metal, glass, ceramic, plastic, hydroxyapatite, treated or untreated bone, a synthetic polymer, a natural substance, a semisynthetic material, and combinations thereof.

4. The method according to claim 3, wherein the material is degradable.

5. The method according to claim 3, wherein the material is non-degradable.

6. The method according to claim 1, wherein the culture medium contains exogenous growth factors, cytokines, lymphokines, hormones, chemokines, interleukins, mitogens, antigens or antigenic fragments thereof, or combination thereof.

7. The method according to claim 6, wherein the culture medium contains cytokines which are selected from the group consisting of interleukin-2, interleukin-4, interleukin-6, interleukin-10, interleukin-7, interleukin-12, flt-3 Ligand, stem cell factor, thrombopoietin, CD40 ligand, BAC-1, L-BCGF, soluble interleukin 6R, and combinations thereof.

8. The method according to claim 6, wherein the culture medium contains an antigen which is selected from the group consisting of a peptide, protein, carbohydrate, glycoprotein, proteoglycan, lipopolysaccharide, nucleic acid, virus, cells, cell fragment, tissue, and combination thereof.

9. The method according to claim 8, wherein the antigen is a tumor antigen.

10. The method according to claim 6, wherein the culture medium contains antigens and antigenic fragments that are presented by antigen presenting cells.

11. The method according to claim 10, wherein the antigen presenting cells are dendritic cells.

12. The method according to claim 6, wherein the culture medium contains antigens or antigenic fragments that are present as a conjugate.

13. The method according to claim 1, wherein the peripheral lymphoid organ cells are B-cells, T-cells, or combination thereof.

14. The method according to claim 13, wherein the peripheral lymphoid organ cells are T-cells which are selected from the group consisting of cytotoxic T-cells, helper T-cells, and combination thereof.

15. The method according to the claim 13, wherein the peripheral lymphoid organ cells are B-cells which are selected from the group consisting of immature B cells, naïve B cells, memory B-cells, B1 cells, B2 cells, plasma cells, and combination thereof.

16. The method according to claim 1, wherein the peripheral lymphoid organ cells are selected from the group consisting of natural killer (NK) cells, dendritic and follicular dendritic cells, granulocytes, macrophages, and stromal cell subsets.

17. The method according to claim 1, wherein the cultured peripheral lymphoid organ cells are selected from the group consisting of spleen cells, lymph node cells, thymus cells, Peyer's patches cells, and combinations thereof.

18. The method according to claim 17, wherein the cultured peripheral lymphoid organ cells are spleen cells.

19. The method according to claim 1, wherein the cultured peripheral lymphoid organ cells express a surface marker selected from the group consisting of CD5, CD23, CD69, CD25, MHC class I or II, CD80/86, CD138, CD38, CD27, CD8, CD4, CD3, CD45-RO, CD45-RA, and combinations thereof.

20. The method according to claim 1, wherein the cultured peripheral lymphoid organ cells fail to express a surface marker selected from the group consisting of CD5, CD23, CD69, CD25, MHC class I or II, CD80/86, CD138, CD38, CD27, CD8, CD4, CD3, CD45-RO, CD45-RA, and combinations thereof.

21. The method according to claim 1, wherein said culturing is carried out without addition of external mitogens.

22. The method according to claim 1 further comprising:
seeding or reseeding the culture medium with peripheral lymphoid organ cells, peripheral lymphoid cells, primary lymphoid organ cells, stem cells, or combinations thereof.

23. The method according to claim 22, wherein the culture medium is reseeded with peripheral lymphoid organ cells selected from the group consisting of spleen cells, lymph node cells, Peyer's patches cells, and combinations thereof.

24. The method according to claim 1, wherein said culturing is carried out with an antigen or antigenic fragment thereof in the culture medium and under conditions effective to produce antigen-specific lymphocytes.

25. The method according to claim 24, wherein said culturing is carried out under conditions effective to permit clonal selection, expansion, and/or affinity maturation of lymphocytes.

26. The method according to claim 24, further comprising:
adding an adjuvant to the cell culture.

27. The method according to claim 24, wherein the antigen is selected from the group consisting of a peptide, protein, carbohydrate, glycoprotein, proteoglycan, lipopolysaccharide, nucleic acid, virus, cells, cell fragment, tissue, and combinations thereof.

28. The method according to claim 24, wherein the antigen is a tumor antigen.

29. The method according to claim 24, wherein the antigen or antigenic fragment are presented by antigen presenting cells.

30. The method according to claim 24, wherein the antigen or antigenic fragment are present as a conjugate.

31. The method according to claim 24, wherein said culturing is carried out under conditions effective to permit the antigen-specific lymphocytes to produce antibodies.

32. The method according to claim 24 further comprising:
immortalizing the antigen-specific lymphocytes.

33. The method according to claim 32, wherein the lymphocytes are B-cells, T-cells, or combinations thereof.

34. The method according to claim 32, wherein said immortalizing is induced.

35. The method according to claim 34, wherein said immortalizing comprises:
fusing the antigen-specific lymphocytes to a cell line under conditions effective to produce a hybridoma cell line.

36. The method according to claim 35 further comprising:
recovering monoclonal antibodies from the hybridoma cell line.

37. The method according to claim 32, wherein said immortalizing is spontaneous.

38. A method of screening for vaccine candidates for efficacy in eliciting an immune response comprising:
culturing peripheral lymphoid organ cells in a container on a three-dimensional scaffolding which is covered or surrounded with culture medium under conditions effective to generate and maintain mature and functional peripheral lymphoid organ cells, wherein said three-dimensional scaffolding allows the peripheral lymphoid organ cells in the culture medium to have cell to cell contact in three dimensions.
adding a vaccine candidate to the container; and
determining whether the vaccine candidate elicits an immune response in the cultured peripheral lymphoid organ cells.

39. The method according to claim 38, wherein the cells are mammalian peripheral lymphoid organ cells.

40. The method according to claim 39, wherein the mammalian peripheral lymphoid organ cells are human cells.

41. The method according to claim 38, wherein the culture medium contains exogenous growth factors, cytokines, lymphokines, hormones, chemokines, interleukins, mitogens, antigens or antigenic fragments thereof, or combinations thereof.

42. The method according to claim 41, wherein the culture medium contains cytokines which are selected from the group consisting of interleukin-2, interleukin-4, interleukin-6, interleukin-10, interleukin-7, interleukin-12, flt-3 Ligand, stem cell factor, thrombopoietin, CD40 ligand, BAC-1, L-BCGF, soluble interleukin 6R, and combinations thereof.

43. The method according to claim 41, wherein the culture medium contains an antigen which is selected from the group consisting of a peptide, protein, glycoprotein, proteoglycan, lipopolysaccharide, nucleic acid, virus, cells, cell fragment, tissue, and combinations thereof.

44. The method according to claim 38, wherein the peripheral lymphoid organ cells are B-cells, T-cells, or combinations thereof.

45. The method according to claim 44, wherein the peripheral lymphoid organ cells are T-celis which are selected from the group consisting of cytotoxic T-cells, helper T-cells, and combinations thereof.

46. The method according to the claim 44, wherein the peripheral lymphoid organ cells are B-cells which are selected from the group consisting of immature B cells, naïve B cells, memory B-cells, B1 cells, B2 cells, plasma cells, and combinations thereof.

47. The method according to claim 38, wherein the cultured peripheral lymphoid organ cells are selected from the group consisting of spleen cells, lymph node cells, thymus cells, Peyer's patches cells, and combinations thereof.

48. The method according to claim 47, wherein the cultured peripheral lymphoid organ cells are spleen cells.

49. The method according to claim 47, wherein the cultured peripheral lymphoid organ cells are lymph node cells.

50. The method according to claim 38, wherein the cultured peripheral lymphoid organ cells express a surface marker selected from the group consisting of CD5, CD23, CD69, CD25, NHC class I or II, CD80/86, CD138, CD38, CD27, CD8, CD4, CD3, CD45-RO, CD45-RA, and combinations thereof.

51. The method according to claim 38, wherein the cultured peripheral lymphoid organ cells fail to express a surface marker selected from the group consisting of CD5, CD23, CD69, CD25, NHC class I or II, CD80/86, CD138, CD38, CD27, CD8, CD4, CD3, CD45-RO, CD45-RA, and combinations thereof.

52. The method according to claim 38, wherein the scaffolding is selected from the group consisting of tangled fibers, porous particles, sponge, sponge-like material, and combinations thereof.

53. The method according to claim 38, wherein the scaffolding is formed from a material selected from the group consisting of a synthetic polymer, a natural substance, a semisynthetic material, and combinations thereof.

54. The method according to claim 53, wherein the material is degradable.

55. The method according to claim 53, wherein the material is non-degradable.

56. The method according to claim 38, wherein said culturing is carried out without addition of external mitogens.

57. The method according to claim 38 further comprising:
seeding or reseeding the culture medium with peripheral lymphoid organ cells, peripheral lymphoid cells, primary lymphoid organ cells, stem cells, or combinations thereof.

58. The method according to claim 57, wherein the culture medium is reseeded with peripheral lymphoid organ cells selected from the group consisting of spleen cells, lymph node cells, Peyer's patches cells, and combinations thereof.

59. A method of identifying genes or proteins which are related to peripheral lymphoid organ cell formation or function which comprises:
culturing peripheral lymphoid organ cells on a three-dimensional scaffolding which is covered or surrounded with culture medium under conditions effective to generate and maintain mature and functional peripheral lymphoid organ cells, wherein said three-dimensional scaffolding allows the cells in the culture medium to have cell to cell contact in three dimensions.
altering one or more culture conditions in a test culture;
determining peripheral lymphoid organ cell number and function in the test culture; and
screening for genes or proteins associated with a change in peripheral lymphoid organ cell number or function in the test culture.

60. The method according to claim 59, wherein cell number is determined by immunohistochemistry, cell morphology, or flow cytometry.

61. The method according to claim 59, wherein said screening for proteins or genes associated with change in peripheral lymphoid cell number or function involves at least one of differential display, RNA arbitrarily primed (RAP)—PCR, or microarray analysis.

62. The method according to claim 59, wherein the peripheral lymphoid organ cells are mammalian peripheral lymphoid organ cells.

63. The method according to claim 62, wherein the mammalian peripheral lymphoid organ cells are human cells.

64. The method according to claim 59, wherein the culture medium contains exogenous growth factors, cytokines, lymphokines, hormones, chemokines, interleukins, mitogens, antigens or antigenic fragments thereof, or combinations thereof.

65. The method according to claim 64, wherein the culture medium contains cytokines which are selected from the group consisting of interleukin-2, interleukin-4, interleukin-6, interleukin-10, interleukin-7, interleukin-12, flt-3 Ligand, stem cell factor, thrombopoietin, CD40 ligand, BAC-1, L-BCGF, soluble interleukin 6R, and combinations thereof.

66. The method according to claim 64, wherein the culture medium contains an antigen which is selected from the group consisting of a peptide, protein, carbohydrate, glycoprotein, proteoglycan, lipopolysaccharide, nucleic acid, virus, cells, cell fragment, tissue, and combinations thereof.

67. The method according to claim 59, wherein the peripheral lymphoid organ cells are B-cells, T-cells, or combinations thereof.

68. The method according to claim 67, wherein the peripheral lymphoid organ cells are T-cells which are selected from the group consisting of cytotoxic T-cells, helper T-cells, and combinations thereof.

69. The method according to the claim 67, wherein the peripheral lymphoid organ cells are B-cells which are selected from the group consisting of immature B cells, naïve B cells, memory B-cells, B1 cells, B2 cells, plasma cells, and combinations thereof.

70. The method according to claim 59, wherein the cultured peripheral lymphoid organ cells are selected from the group consisting of spleen cells, lymph node cells, thymus cells, Peyer's patches cells, and combinations thereof.

71. The method according to claim 70, wherein the cultured peripheral lymphoid organ cells are spleen cells.

72. The method according to claim 40, wherein the cultured peripheral lymphoid organ cells are lymph node cells.

73. The method according to claim 59, wherein the cultured peripheral lymphoid organ cells express a surface marker selected from the group consisting of CD5, CD23, CD69, CD25, MHC class I or II, CD80/86, CD138, CD38, CD27, CD8, CD4, CD3, CD45-RO, CD45-RA, and combinations thereof.

74. The method according to claim 59, wherein the cultured peripheral lymphoid organ cells fail to express a surface marker selected from the group consisting of CD5, CD23, CD69, CD25, MHC class I or II, CD80/86, CD138, CD38, CD27, CD8, CD4, CD3, CD45-RO, CD45-RA, and combinations thereof.

75. The method according to claim 59, wherein the scaffolding is selected from the group consisting of tangled fibers, porous particles, sponge, sponge-like material, and combinations thereof.

76. The method according to claim 59, wherein the scaffolding is formed from a material selected from the group consisting of a synthetic polymer, a natural substance, a semisynthetic material, and combinations thereof.

77. The method according to claim 76, wherein the material is degradable.

78. The method according to claim 76, wherein the material is non-degradable.

79. The method according to claim 59, wherein said culturing is carried out without addition of external mitogens.

80. The method according to claim 59 further comprising:
seeding or reseeding the culture medium with peripheral lymphoid organ cells, peripheral lymphoid cells, primary lymphoid organ cells, stem cells, or combinations thereof.

81. The method according to claim 80, wherein the culture medium is reseeded with peripheral lymphoid organ cells selected from the group consisting of spleen cells, lymph node cells, Peyer's patches cells, and combinations thereof.

82. A method of screening for drugs effecting peripheral lymphoid organ cell generation, maturation or function which comprises:
culturing peripheral lymphoid organ cells in a container on a three-dimensional scaffolding which is covered or surrounded with culture medium under conditions effective to generate and maintain mature and functional peripheral lymphoid organ cells, wherein said three-dimensional scaffolding allows the cells in the culture medium to have cell to cell contact in three dimensions.
adding a test compound to the container,
removing cultured cells from the container; and
determining the test compound's ability to effect lymphoid cell generation, maturation or function.

83. The method according to claim 82, wherein the ability of a test compound to effect peripheral lymphoid cell generation, maturation or function is determined by lymphoid peripheral cell count, immunohistochemistry, flow cytometry, or a combination thereof.

84. The method according to claim 82, wherein the test compound stimulates peripheral lymphoid organ cell maturation.

85. The method according to claim 82, wherein the test compound inhibits peripheral lymphoid organ cell maturation.

86. The method according to claim 82, wherein the cells are mammalian peripheral lymphoid organ cells.

87. The method according to claim 86, wherein the mammalian peripheral lymphoid organ cells are human cells.

88. The method according to claim 82, wherein the culture medium contains exogenous growth factors, cytokines, lymphokines, hormones, chemokines, interleukins, mitogens, antigens or antigenic fragments thereof, or combinations thereof.

89. The method according to claim 88, wherein the culture medium contains cytokines which are selected from the group consisting of interleukin-2, interleukin-4, interleukin-6, interleukin-10, interleukin-7, interleukin-12, flt-3 Ligand, stem cell factor, thrombopoietin, CD40 ligand, BAC-1, L-BCGF, soluble interleukin 6R, and combinations thereof.

90. The method according to claim 88, wherein the culture medium contains an antigen which is selected from the group consisting of a peptide, protein, carbohydrate, glycoprotein, proteoglycan, lipopolysaccharide, nucleic acid, virus, cells, cell fragment, tissue, and combinations thereof.

91. The method according to claim 82, wherein the peripheral lymphoid organ cells are B-cells, T-cells, or combinations thereof.

92. The method according to claim 91, wherein the peripheral lymphoid organ cells are T-cells which are selected from the group consisting of cytotoxic T-cells, helper T-cells, and combinations thereof.

93. The method according to the claim 91, wherein the peripheral lymphoid organ cells are B-cells which are selected from the group consisting of immature B cells, naïve B cells, memory B-cells, B 1 cells, B2 cells, plasma cells, and combinations thereof.

94. The method according to claim 82, wherein the cultured peripheral lymphoid organ cells are selected from the group consisting of spleen cells, lymph node cells, thymus cells, Peyer's patches cells, and combinations thereof.

95. The method according to claim 94, wherein the cultured peripheral lymphoid organ cells are spleen cells.

96. The method according to claim 94, wherein the cultured peripheral lymphoid organ cells are lymph node cells.

97. The method according to claim 82, wherein the cultured peripheral lymphoid organ cells express a surface marker selected from the group consisting of CD5, CD23, CD69, CD25, MHC class I or II, CD80/86, CD138, CD38, CD27, CD8, CD4, CD3, CD45-RO, CD45-RA, and combinations thereof.

98. The method according to claim 82, wherein the cultured peripheral lymphoid organ cells fail to express a surface marker selected from the group consisting of CD5, CD23, CD69, CD25, MHC class I or II, CD80/86, CD138, CD38, CD27, CD8, CD4, CD3, CD45-RO, CD45-RA, and combinations thereof.

99. The method according to claim 82, wherein the scaffolding is selected from the group consisting of tangled fibers, porous particles, sponge, sponge-like material, and combinations thereof.

100. The method according to claim 82, wherein the scaffolding is formed from a material selected from the group consisting of a synthetic polymer, a natural substance, a semisynthetic material, and combinations thereof.

101. The method according to claim 100, wherein the material is degradable.

102. The method according to claim 100, wherein the material is non-degradable.

103. The method according to claim 82, wherein said culturing is carried out without addition of external mitogens.

104. The method according to claim 82 further comprising:
seeding or reseeding the culture medium with peripheral lymphoid organ cells, peripheral lymphoid cells, primary lymphoid organ cells, stem cells, or combinations thereof.

105. The method according to claim 104, wherein the culture medium is reseeded with peripheral lymphoid organ cells selected from the group consisting of spleen cells, lymph node cells, Peyer's patches cells, and combinations thereof.

106. A method of treating a patient for a disease condition, wherein the method comprises:
culturing peripheral lymphoid organ cells on a three-dimensional scaffolding which is covered or surrounded with culture medium under conditions effective to generate and maintain mature and functional peripheral lymphoid organ cells, wherein said three-dimensional scaffolding allows cells in the culture medium to have cell to cell contact in three dimensions; and
administering to a patient an effective amount of peripheral lymphoid organ cells produced in the three dimensional cell culture system, thereby treating the patient for the disease condition.

107. The method according to claim 106, wherein the mammalian peripheral lymphoid organ cells are B-cells, T-cells, or combinations thereof.

108. The method according to claim 107, wherein the mammalian peripheral lymphoid organ cells are T-cells.

109. The method according to claim 106, wherein said culturing is carried out with an antigen or antigenic fragment thereof in the culture medium and under conditions effective to produce antigen-specific lymphocytes.

110. The method according to claim 109, further comprising:
adding an adjuvant to the cell culture.

111. The method according to claim 109, wherein the antigen is selected from the group consisting of a peptide, a protein, a glycoprotein, a proteoglycan, a lipopolysaccharide, a virus, cell, a cell fragment, tissue, and combinations thereof.

112. The method according to claim 111, wherein the antigen is a tumor antigen.

113. The method according to claim 109, wherein said culturing is carried out under conditions effective to permit the antigen-specific lymphocytes to produce antibodies.

114. The method according to claim 109 further comprising:
immortalizing the antigen-specific lymphocytes.

115. The method according to claim 114, wherein said immortalizing is induced.

116. The method according to claim 115, wherein said immortalizing comprises:
fusing the antigen-specific lymphocytes to a cell line under conditions effective to produce a hybridoma cell line.

117. A method of treating a patient for a disease condition wherein the method comprises:
administering to a patient an effective amount of an antibody produced according to claim 31, thereby treating the patient for the disease condition.

118. A method of treating a patient for a disease condition, wherein the method comprises:
administering to a patient an effective amount of an antibody produced according to claim 36, thereby treating the patient for the disease condition.

119. A method for effecting gene expression of peripheral lymphoid organ cells, said method comprising:
culturing peripheral lymphoid organ cells on a three-dimensional scaffolding which is covered or surrounded with culture medium under conditions effective to generate and maintain mature and functional peripheral lymphoid organ cells, wherein said three-dimensional scaffolding allows cells in the culture medium to have cell to cell contact in three dimensions; and
transforming or transducing the cultured peripheral lymphoid organ cells with a desired gene, thereby effecting gene expression of the peripheral lymphoid organ cells.

120. A method of treating a patient for a disease condition, said method comprising:
administering to a patient an effective amount of the transformed or transduced the cultured peripheral lymphoid organ cells according to claim 119, thereby treating the patient for the disease condition.
